(19) United States
(12) Patent Application Publication
(10) Pub. No.: US 2026/0327247 A1
(43) Pub. Date: Oct. 22, 2026

(54) C-N coupling process with Cu2(salben)2 complexes

(71) Applicant: Hoffmann-La Roche Inc., Little Falls, NJ (US)

(72) Inventors: Serena Maria Fantasia, Saint-Louis (FR); Lorenzo Marchi, Castelnuovo Rangone (IT); Luca Rigamonti, Modena (IT); Markus Steiner, Basel (SW)

(73) Assignee: Hoffmann-La Roche Inc., Little Falls, NJ (US)

(21) Appl. No.: 19/444,608

(22) Filed: Jan. 9, 2026

Related U.S. Application Data
(63) Continuation of application No. PCT/EP2024/069763, filed on Jul. 12, 2024.

(30) Foreign Application Priority Data
Jul. 14, 2023 (EP) 23185471.2

(51) Int. Cl. C07F 1/08; C07C 209/10; C07C 231/08; C07C 303/40; C07D 207/325; C07D 231/12; C07D 233/58; C07D 249/06; C07D 401/04; C07D 498/04

(52) U.S. Cl. CPC C07F 1/08; C07C 209/10; C07C 231/08; C07C 303/40; C07D 207/325; C07D 231/12; C07D 233/58; C07D 249/06; C07D 401/04; C07D 498/04

(57) ABSTRACT

The invention relates to a process for the coupling of an aryl halogenide with an N-nucleophile compound which is characterized in that the coupling takes place in the presence of a dinuclear copper(II) complex bearing substituted salben-type ligands having the formula I

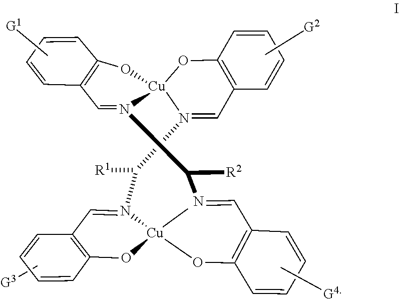

Also described are novel dinuclear copper(II) complex bearing substituted salben-type ligands within the scope of formula I

The invention relates to a novel process for the coupling of aryl halogenides with an N-nucleophile compound in the presence of dinuclear copper(II) complexes bearing substituted salben-type ligands of the formula I

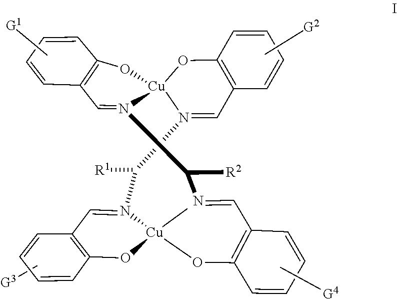

wherein R¹ and R² are the same or different and are optionally substituted C₁₋₁₂-alkyl, aryl or heteroaryl or hydrogen, and
G¹, G², G³ and G⁴ are the same or different and stand for hydrogen or one or more substituents selected from C₁₋₁₂-alkyl, C₁₋₁₂-alkoxy, halogen C₁₋₁₂-alkyl, halogen, mono- or di-C₁₋₁₂-alkyl amino, carboxyl, C₁₋₁₂ alkoxy carbonyl or nitro or G¹, G², G³ and G⁴, independently of each other, together with the phenyl ring they are attached to form a fused aryl ring with two or three rings, which are optionally substituted with one or more substituents selected from C₁₋₆-alkyl, C₁₋₆-alkoxy, halogen, mono- or di-C₁₋₆-alkyl amino or nitro.

The inventions also relates to novel dinuclear copper(II) complexes bearing substituted salben-type ligands of the formula I, wherein
R¹ and R² are the same and are substituents of the formula IIa, IIb or IIc

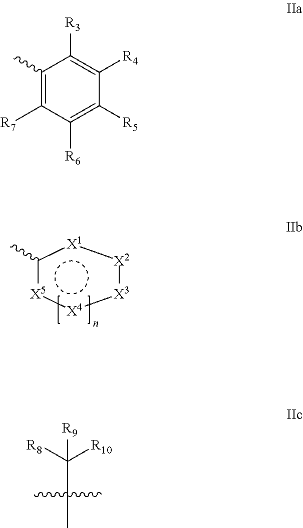

wherein
R³, R⁴, R⁵, R⁶ and R⁷ are the same or different and are hydrogen, C₁₋₁₂-alkyl, C₁₋₁₂-alkoxy, halogen, halogen-C₁₋₁₂-alkyl, mono- or di-C₁₋₁₂-alkyl amino, nitro or R³ and R⁴ or R⁴ and R⁵ or R⁵ and R⁶ or R⁶ and R⁷ together with the phenyl ring they are attached to form a fused aryl ring with two or three rings, which are optionally substituted with one or more substituents selected from C₁₋₆-alkyl, C₁₋₆-alkoxy, halogen, mono- or di-C₁₋₆-alkyl amino or nitro.
X¹, X², X³, X⁴ and X⁵ are the same or different and represent at least one heteroatom selected from nitrogen, oxygen or sulphur;
n is 0 or 1;
R⁸, R⁹ and R¹⁰ are the same or different and are hydrogen, C₁₋₁₂-alkyl, halogen C₁₋₁₂-alkyl, hydroxy, hydroxy-C₁₋₁₂-alkyl, halogen or R⁸ and R⁹ together form a saturated carbocycle or a heterocycle or R⁸, R⁹ and R¹⁰ together form an unsaturated carbocycle,
with the proviso that compounds with R¹ and R² being hydrogen, or R¹ and R² having the formula IIa and wherein R³, R⁴, R⁶ and R are hydrogen, while R⁵ is hydrogen, chlorine, methyl, or nitro, are excluded.

The object of the present invention was to provide an alternative method for the C—N-coupling, i.e. the coupling of an aryl halogenide with an N-nucleophile with a dinuclear copper(II) complex bearing substituted salben-type ligands of the formula I and to provide novel dinuclear copper(II) complex bearing substituted salben-type ligands of the formula I capable to catalyse the coupling.

The object could be reached with the process as outlined below, which comprises the coupling of an aryl halogenide with an N-nucleophile compound in the presence of a dinuclear copper(II) complex bearing substituted salben-type ligands having the formula I.

The following definitions are set forth to illustrate and define the meaning and scope of the various terms used to describe the invention herein.

The term “C₁₋₁₂-alkyl” relates to a branched or straight-chain monovalent saturated aliphatic hydrocarbon radical of one to twelve carbon atoms, preferably one to eight, more preferably one to four carbon atoms. This term is further exemplified by radicals as methyl, ethyl, n-propyl, i-propyl, n-butyl, i-butyl, s-butyl or t-butyl, pentyl, or hexyl, heptyl, octyl, nonyl, decyl, undecyl and dodecyl and its isomers.

The term “C₁₋₁₂-alkoxy” refers to a C₁₋₁₂-alkyl group as defined above to which an oxygen atom is attached. Typical representatives are methoxy or ethoxy.

The term “mono- or di-C₁₋₁₂-alkyl amino” refers to an amino group which is mono- or di-substituted by a C₁₋₁₂-alkyl group as defined above. Typical examples are N-methylamino, N-ethylamino, N,N-dimethylamino or N,N-diethylamino.

The term “halogen” refers to fluorine, chlorine, bromine or iodine, preferably to fluorine and chlorine, but in regard to the C—N coupling particularly to bromine and iodine.

The term “halogen-C₁₋₁₂-alkyl” refers to a C₁₋₁₂-alkyl group as defined above which is substituted with one or more halogens, such as trichloromethyl, 1,1-dichloroethyl or 1,1-di-chlorooctyl.

The term “aryl” relates to an aromatic carbon ring such as to the phenyl, naphthyl ring anthracenyl or phenanthrenyl ring, preferably the phenyl ring.

The term “heteroaryl” refers to an aromatic 5 to 6 membered monocyclic ring or 9 to 10 membered bicyclic ring which can comprise 1, 2 or 3 heteroatoms selected from nitrogen, oxygen and/or sulphur, such as pyridyl, pyridinyl, pyrazolyl, pyrimidinyl, benzoimidazolyl, quinolinyl and isoquinolinyl, thienyl, furyl, pyrrolyl, isoxazolyl, oxazolyl, thiazolyl or imidazolyl.

The term “optionally substituted” refers to one or more substituents selected from C₁₋₁₂-alkyl, C₁₋₁₂-alkoxy, halogen, halogen C₁₋₁₂-alkyl, mono- or di-C₁₋₁₂-alkyl amino or nitro.

The spiral bond [symbol]
stands for “ [symbol] ” or for “ [symbol] ” and thus indicating chirality of the molecule, but also for mixtures of the enantiomers.

Whenever a chiral carbon is present in a chemical structure, it is intended that all stereoisomers associated with that chiral carbon are encompassed by the structure as pure stereoisomers as well as mixtures thereof.

### Process:

As outlined above the invention relates to a novel process for the coupling of aryl halogenides with an N-nucleophile compound in the presence of dinuclear copper(II) complexes bearing substituted salben-type ligands of the formula I.

### Aryl Halogenide:

Suitable aryl halogenides can be defined with the formula III

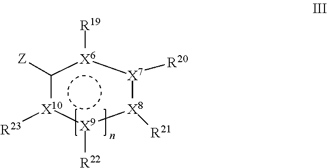

wherein
X⁶, X⁷, X⁸, X⁹ and X¹⁰ are the same or different and represent carbon or at least one heteroatom selected from nitrogen, oxygen or sulphur;
R¹⁹, R²⁰, R²¹, R²² and R²³ are the same or different and are hydrogen, C₁₋₁₂-alkyl, C₂₋₁₂-alkenyl, C₂₋₁₂-alkynyl, C₁₋₁₂-alkoxy, halogen, halogen-C₁₋₁₂-alkyl, mono- or di-C₁₋₁₂-alkyl amino or cyano, or R¹⁹ and R²⁰ or R²⁰ and R²¹ or R²¹ and R²² or R²² and R²³ together with the ring they are attached to form a fused aryl ring with two or three rings, which are optionally substituted with one or more substituents selected from C₁₋₁₂-alkyl, C₁₋₁₂-alkoxy, halogen, mono- or di-C₁₋₆-alkyl amino or nitro;
n is 0 or 1 and
Z is a halogen atom.

In preferred aryl halogenides
X⁶, X⁷, X⁸, X⁹ and X¹⁰ are the same or different and represent carbon or one nitrogen atom;
R¹⁹, R²⁰, R²¹, R²² and R²³ are the same or different and are hydrogen, methyl, methoxy or cyano;
n is 1 and
Z is chlorine, bromine or iodine, preferably bromine or iodine.

The aryl halogenides of the formula III are as a rule commercially available or they can be synthesized following procedures well known by the skilled in the art.

### N-nucleophile:

Suitable N-nucleophile can be selected from compounds of the formulae

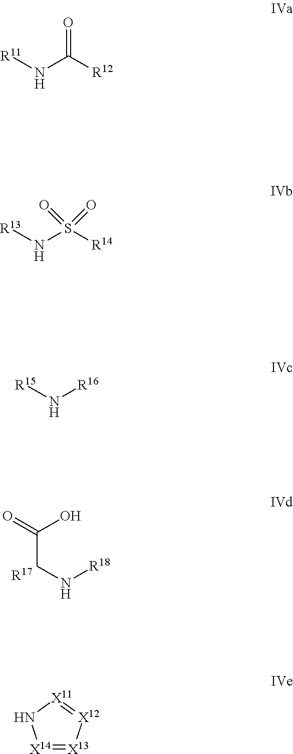

wherein
R¹¹, R¹², R¹³, R¹⁴, R¹⁵, R¹⁶, R¹⁷ and R¹⁸ are the same or different and are hydrogen, C₁₋₁₂-alkyl, C₁₋₁₂-alkoxy, aryl, halogen, halogen-C₁₋₁₂-alkyl, nitro, hydroxy, mono- or di-C₁₋₁₂-alkyl amino or R¹⁵ and R¹⁶ together with the nitrogen they are attached to form a saturated 5- or 6-member heterocycle;
X¹¹, X¹², X¹³ and X¹⁴ are the same or different and represent a carbon atom optionally substituted with C₁₋₁₂-alkyl, aryl-C₁₋₁₂-alkyl, C₁₋₁₂-alkoxy, halogen, halogen-C₁₋₁₂-alkyl and at least one further heteroatom selected from nitrogen, oxygen or sulphur.

Preferred N-nucleophiles have the formula IVc, IVd, IVe, wherein
R¹⁵, R¹⁶, R¹⁷ and R¹⁸ are the same or different and are hydrogen, C₁₋₆-alkyl, aryl, or R⁵ and R¹⁶ together with the nitrogen they are attached to form a saturated heterocycle 5- or 6-member heterocycle;
X¹¹, X¹², X¹³ and X¹⁴ are the same or different and represent a carbon atom optionally substituted with C₁₋₁₂-alkyl, aryl-C₁₋₁₂-alkyl, and at least one further heteroatom selected from nitrogen, oxygen or sulphur.

### Catalysts:

The process of the present invention takes place in the presence of a dinuclear copper(II) complexes bearing substituted salben-type ligands having the formula I

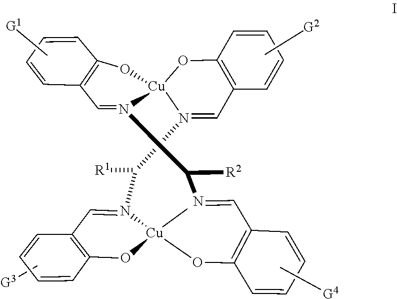

wherein R¹ and R² are the same or different and are optionally substituted C₁₋₁₂-alkyl, aryl or heteroaryl, or hydrogen and
G¹, G², G³ and G⁴ are the same or different and stand for hydrogen or one or more substituents selected from C₁₋₁₂-alkyl, C₁₋₁₂-alkoxy, halogen, halogen C₁₋₁₂-alkyl, mono- or di-C₁₋₁₂-alkyl amino, carboxyl, C₁₋₁₂-alkoxy carbonyl or nitro or G¹, G², G³ and G⁴, independently of each other, together with the phenyl ring they are attached to form a fused aryl ring with two or three rings, which are optionally substituted with one or more substituents selected from C₁₋₆-alkyl, C₁₋₆-alkoxy, halogen, mono- or di-C₁₋₆-alkyl amino or nitro.

In a preferred embodiment G¹, G², G³ and G⁴ are the same or different and stand for hydrogen or one or more substituents selected from methyl, ethyl, t-butyl, methoxy, ethoxy, fluorine, chlorine, bromine, trifluoromethyl, N.N-dimethylamino, N.N-diethylamino, carboxyl, methoxy carbonyl, ethoxy carbonyl, i-propoxy carbonyl, n-butoxy carbonyl, t-butoxy carbonyl, nitro or G¹, G², G³ and G⁴, independently of each other, together with the phenyl ring they are attached to form a fused aryl ring.

In a preferred embodiment R¹ and R² are the same and are substituents of the formula IIa, IIb or IIc

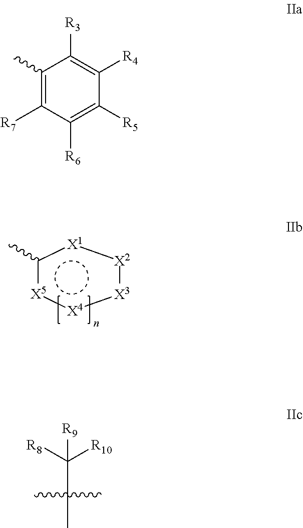

wherein
R³, R⁴, R⁵, R⁶ and R⁷ are the same or different and are hydrogen, C₁₋₁₂-alkyl, C₁₋₁₂-alkoxy, halogen, halogen-C₁₋₁₂-alkyl, mono- or di-C₁₋₁₂-alkyl amino, nitro or R³ and R⁴ or R⁴ and R⁵ or R⁵ and R⁶ or R⁶ and R⁷ together with the phenyl ring they are attached to form a fused aryl ring with two or three rings, which are optionally substituted with one or more substituents selected from C₁₋₆-alkyl, C₁₋₆-alkoxy, halogen, mono- or di-C₁₋₆-alkyl amino or nitro,
X¹, X², X³, X⁴ and X⁵ are the same or different and represent at least one heteroatom selected from nitrogen, oxygen or sulphur;
n is 0 or 1;
R⁸, R⁹ and R¹⁰ are the same or different and are hydrogen, C₁₋₁₂-alkyl, halogen C₁₋₁₂-alkyl, hydroxy, hydroxy-C₁₋₁₂-alkyl, halogen or R⁸ and R⁹ together form a saturated carbocycle or a heterocycle or R⁸, R⁹ and R¹⁰ together form an unsaturated carbocycle.

Preferred substituents of formula IIa are those wherein R³, R⁴, R⁵, R⁶ and R⁷ are the same or different and are hydrogen, methyl, ethyl, t-butyl, methoxy, ethoxy, chlorine, bromine, trifluoromethyl, N.N-dimethylamino, N.N-diethylamino, nitro or R³ and R⁴ or R⁴ and R⁵ or R⁵ and R⁶ or R⁶ and R⁷ together with the phenyl ring they are attached to form a fused aryl ring selected from 2-naphthalene, anthracene or phenanthrene

In particular, examples of formula IIa
R³ to R⁷ are hydrogen, or
R³ is methyl, methoxy, chlorine or nitro and R⁴ to R⁷ are hydrogen, or
R⁵ is methyl, t-butyl, methoxy, chlorine, nitro or N,N-dimethylamino and R³ and R⁴ and R⁶ and R⁷ are hydrogen, or
R³ and R are methoxy or chlorine and R⁴, R⁶ and R⁷ are hydrogen, or
B³ and R are methoxy or chlorine and R⁴ to R⁶ are hydrogen, or
B³, R⁵ and R are methoxy and R⁴ to R⁶ are hydrogen.

Preferred substituents of formula IIb can be selected from

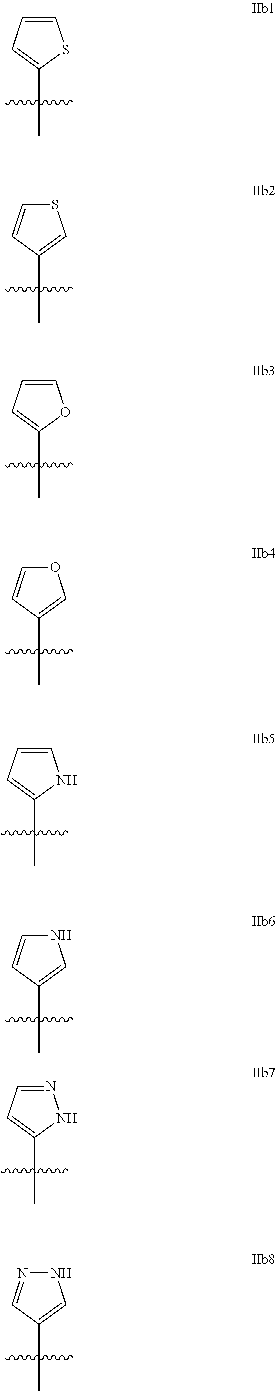

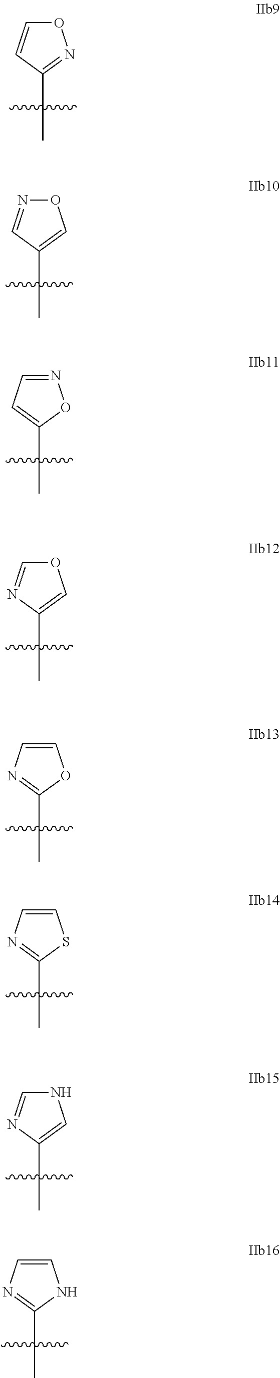

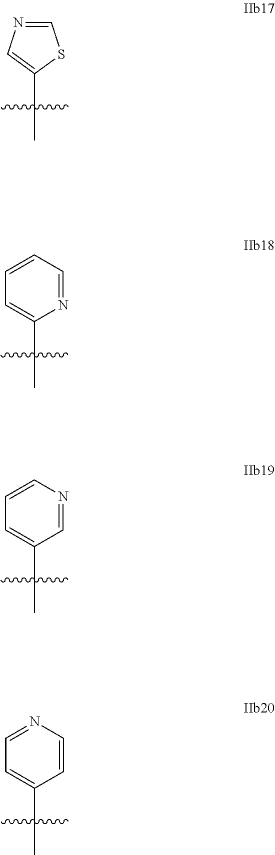 [SMILES: *c1cncs1 ; *c1ccccn1 ; *c1cccnc1 ; *c1ccncc1]

Particularly preferred substituents are of the formula IIb1, IIb2, IIb14, IIb17.

Preferred substituents of formula IIc are those wherein R⁸, R⁹ and R¹⁰ are the same or different and are hydrogen, hydroxy, hydroxy-C₁₋₄-alkyl or R⁸ and R⁹ together form a heterocycle or R⁸, R⁹ and R¹⁰ together form an unsaturated carbocycle.

Particular examples of substituents of formulas IIc are:

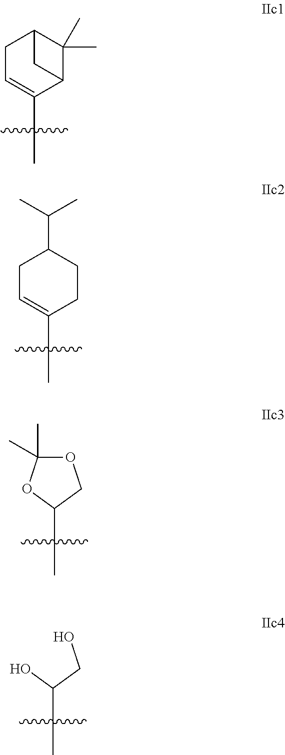

Preferably they are selected from formula IIc1 and IIc2

The substituents IIa and IIc are most preferred.

Particularly preferred catalysts of the formula IIa are those wherein,
R³, R⁴, R⁵, R⁶ and R⁷ are hydrogen, or R³ is chlorine or methoxy and R⁴, R⁵, R⁶ and R⁷ are hydrogen, or R⁵ is chlorine or methoxy and R³, R⁴, R⁶ and R⁷ are hydrogen, or R³ and R⁷ are methoxy and R⁴, R⁵ and R⁶ are hydrogen.

Particularly preferred catalysts of the formula IIc are those with the formula IIc1.

The most preferred catalysts are listed below.

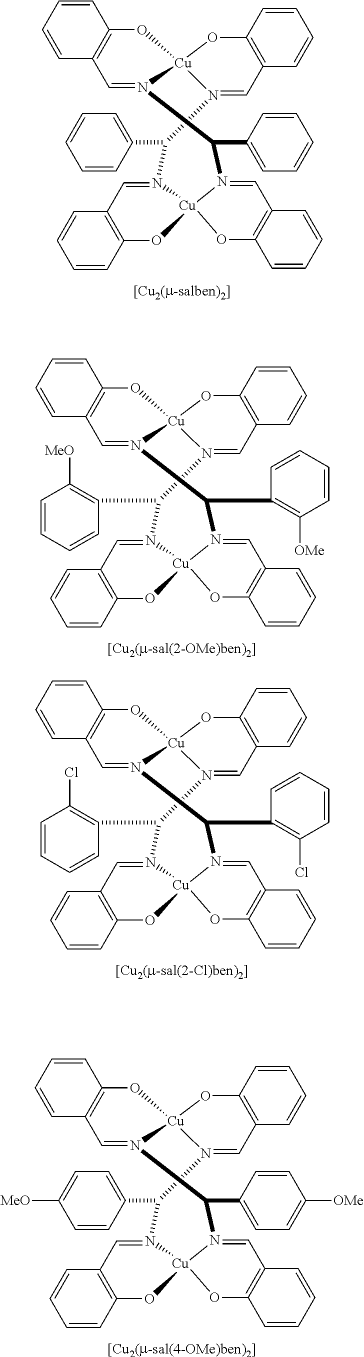

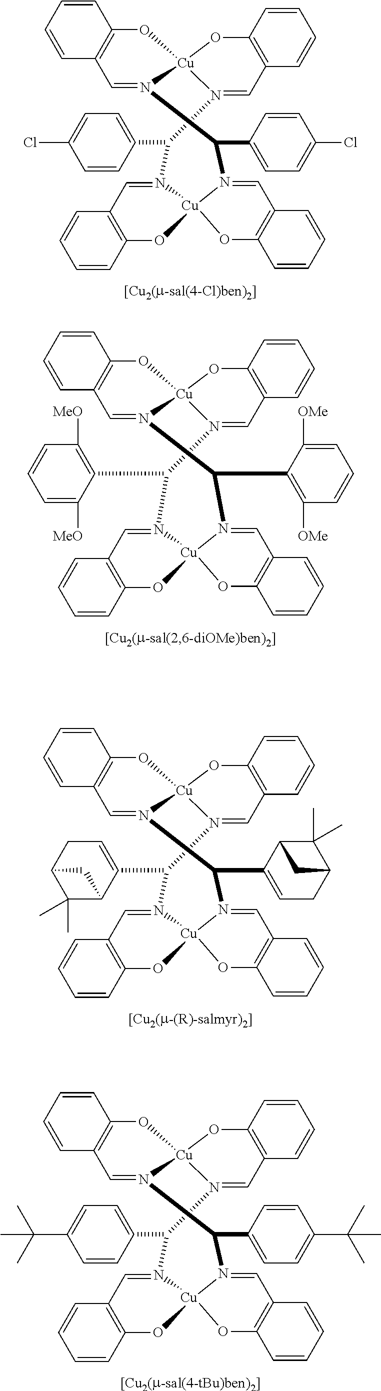

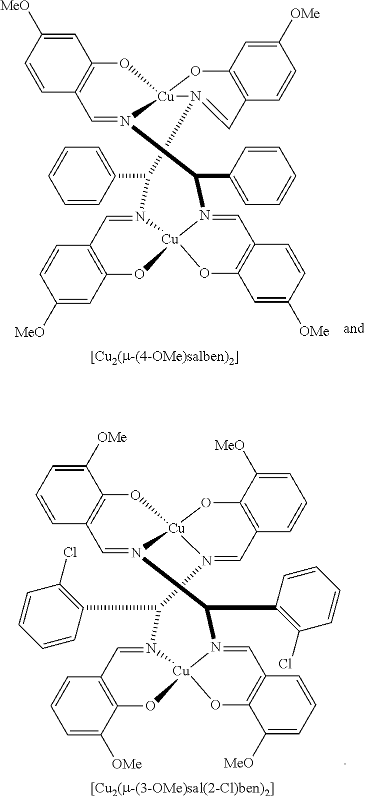

Dinuclear copper(II) complex bearing substituted salben-type ligands having the formula I

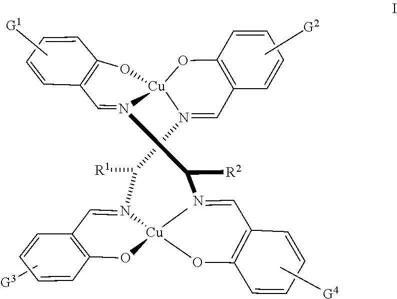

wherein
G¹, G², G³ and G⁴ are the same or different and stand for hydrogen or one or more substituents selected from C₁₋₁₂-alkyl, C₁₋₁₂₆-alkoxy, halogen, halogen C₁₋₁₂-alkyl, mono- or di-C₁₋₁₂-alkyl amino, carboxyl, C₁₋₁₂-alkoxy carbonyl or nitro or G¹, G², G³ and G⁴, independently of each other, together with the phenyl ring they are attached to form a fused aryl ring with two or three rings, which are optionally substituted with one or more substituents selected from C₁₋₆-alkyl, C₁₋₆-alkoxy, halogen, mono- or di-C₁₋₆-alkyl amino or nitro;
R¹ and R² are the same and are substituents of the formula IIa, IIb or IIc

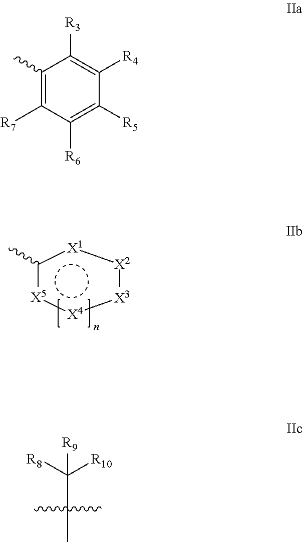

R³, R⁴, R⁵, R⁶ and R⁷ are the same or different and are hydrogen, C₁₋₁₂-alkyl, C₁₋₁₂-alkoxy, halogen, halogen-C₁₋₁₂-alkyl, mono- or di-C₁₋₁₂-alkyl amino, nitro or R³ and R⁴ or R⁴ and R⁵ or R⁵ and R⁶ or R⁶ and R⁷ together with the phenyl ring they are attached to form a fused aryl ring with two or three rings, which are optionally substituted with one or more substituents selected from C₁₋₆-alkyl, C₁₋₆-alkoxy, halogen, mono- or di-C₁₋₆-alkyl amino or nitro.
X¹, X², X³, X⁴ and X⁵ are the same or different and represent at least one heteroatom selected from nitrogen, oxygen or sulphur;
n is 0 or 1;
R⁸, R⁹ and R¹⁰ are the same or different and are hydrogen, C₁₋₁₂-alkyl, halogen C₁₋₁₂-alkyl, hydroxy, hydroxy-C₁₋₁₂-alkyl, halogen or R⁸ and R⁹ together form a saturated carbocycle or a heterocycle or R⁸, R⁹ and R¹⁰ together form an unsaturated carbocycle,
with the proviso that compounds with R¹ and R² being hydrogen, or R¹ and R² having the formula Ila and wherein R R⁴, R⁶ and R⁷ are hydrogen while R⁵ is hydrogen, chlorine, methyl, or nitro, are excluded.

The excluded compounds have been disclosed by
a) Alessandro Pasini, Francesco Demartin, Olivo Piovesana, Brunetto Chiari, Antonio Cinti and Ornella Crispu, J. Chem. Soc., Dalton Trans. 2000, 3467-3472, DOI: 10.1039/b003825n

The novel dinuclear copper(II) complex bearing substituted salben-type ligands having the formula I are novel and therefore constitute a particular embodiment of the present invention.

With the exception for the known compounds, the preferences and particular examples as outlined before likewise apply for the novel dinuclear copper(II) complex bearing substituted salben-type ligands.

### Preparation of Catalysts

The preparation of the catalyst can be accomplished according to the scheme below:

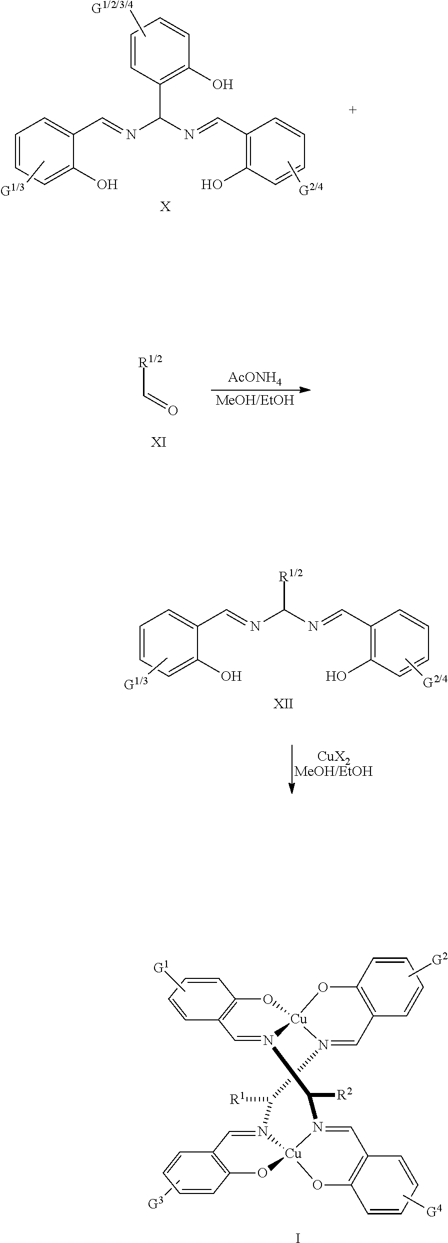

The H₂salben-type derivatives of formula XII can be prepared by reacting an N,N′-bis-(salicylidene)-2-hydroxyphenylmethanediamine derivative of formula X with the appropriate aldehyde of formula X1 in the presence of ammonium acetate a or ammonia and a polar protic solvent such as methanol or ethanol at temperatures from −10 °C to 40 °C under stirring for a period of time from 3 h to 120 h. The resulting ligand can be isolated by filtration, washing with polar protic solvent, and dried under vacuum.

The formation of the dinuclear copper(II) complex bearing substituted salben-type ligands having the formula I can then happen by adding a copper(II) salt CuX₂, preferably the acetate monohydrate, to the suspended H₂salben-type ligand of formula XII in a polar protic solvent, preferably methanol or ethanol, in the presence of tertiary anine, preferably triethylamine at temperatures from −10 °C to 40 °C under stirring for a period of time from W h to 24 h. The resulting complex can be isolated by filtration, washing with polar protic solvent, and dried under vacuum.

### C—N Coupling Process:

The C—N coupling process i.e. the coupling of aryl halogenides with an N-nucleophile compound in the presence of dinuclear copper(II) complexes bearing substituted salben-type ligands of the formula I is advantageously performed in the presence of a suitable base.

The base can be selected from alkali salts of mineral acids or organic acids, such as caesium carbonate, potassium phosphate, potassium carbonate or sodium acetate, or from alkali alcoholates such as sodium- or potassium t-butylate.

The preferred bases are caesium carbonate and potassium phosphate.

As a rule the base is applied in equivalents: from 1 to 5, preferably from 1.5 to 4.

Furthermore, the reaction takes place in the presence of an organic solvent which can be selected from aliphatic alcohols, such as methanol, ethanol, t-amylalcohol, N.N-dimethylformamide, tetrahydrofuran, acetonitrile, toluene or dimethylsulfoxide.

Preferred organic solvents are acetonitrile, tetrahydrofuran and dimethylsulfoxide,

The equivalent aryl halogenide in relation to 1 equivalent of the N-nucleophile, may vary dependent on the halogenide, but typically ranges from 0.2 to 3.0:1.0, preferably 0.3 to 2.5:1.0.

The reaction temperature is as rule held between 20 °C and 200 °C, preferably between 50 °C and 100 °C, more preferably between 70 °C and 90 °C.

The catalyst loading in mol %, referred to the limiting reagent, which can be the N-nucleophile or the aryl halogenide, can vary between 0.1 mol % and 5.0 mol %, preferably between 0.25 mol % and 3.0 mol %.

After a reaction time of 4 h to 24 h, preferably of 8 h to 18 h, the resulting aryl amine can be isolated by filtering off the catalyst and by evaporating the organic phase. The resulting crude aryl amine can further be purified via chromatography.

### EXAMPLES

### Abbreviations

RT=room temperature
MeOH=methanol
Et₂O=diethyl ether
iPr₂O=diisopropyl ether
Et₃N=triethylamine
EtOH=ethanol
MeCN=acetonitrile
DMSO=dimethylsulfoxide
THF=tetrahydrofuran
DCM=dichloromethane

### 1. Activity of Copper(II) Complexes as Catalysts in C—N Coupling Reactions

### 1.1 Optimized Conditions

The coupling of aryl iodide and pyrazole can be achieved by reaction with 0.5 mol % [Cu₂(μ-salben)₂] catalyst in the presence of 2 equiv of Cs₂CO₃ in acetonitrile (MeCN) at 82 °C. After 16 h full conversion is observed, and the product can be isolated in 83% yield.

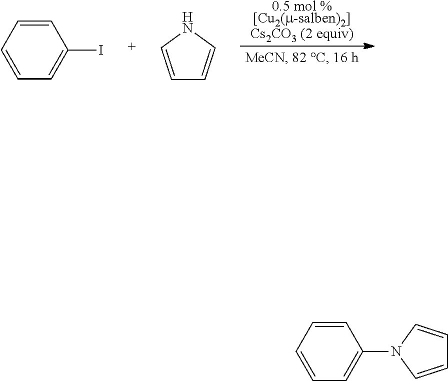

### 1.2 Optimization Studies

TABLE 1
Optimization of temperature and catalyst loading.

| | | Cu source | | | |
|---|---|---|---|---|---|
| | Cu source/ | loading | | t | Yield |
| Entry | ligand | [mol %]^{[a]} | T [°C] | [h] | [%]^{[b]} |
| 1 | [Cu₂(μ- | 0.5 | 82 | 16 | 93 (83) |
| | salben)₂] | | | | |
| 2 | [Cu₂(μ- | 0.5 | RT | 72 | 13 |
| | salben)₂] | | | | |
| 3 | [Cu₂(μ- | 0.25 | 82 | 16 | 80 |
| | salben)₂] | | | | |
| 4 | [Cu₂(μ- | 0.25 | 70 | 16 | 46 |
| | salben)₂] | | | | |
| 5 | [Cu₂(μ- | 0.25 | 60 | 16 | 30 |
| | salben)₂] | | | | |
| 6 | [Cu₂(μ- | 0.25 | 50 | 16 | 16 |
| | salben)₂] | | | | |

General conditions: iodobenzene (215 μL, 1.93 mmol, 1.5 equiv), pyrrole (90 μL, 1.29 mmol, 1 equiv), CS₂CO₃ (839.2 mg, 2.58 mmol, 2 equiv), [Cu₂(μ-salben)₂] in 1 mL of MeCN.^{[a]} The catalyst loading refers to the amount of pyrrole.^{[b] 1}H NMR yield, isolated yields in brackets.

TABLE 2
Screening of bases in MeCN.

| | [Cu₂(μ- | | | |
|---|---|---|---|---|
| | salben)₂] | | | |
| | loading | | | |
| Entry | [mol %]^{[a]} | Base | t [h] | Yield [%]^{[b]} |
| 7 | 0.25 | K₃PO₄ | 16 | 40 |
| 8 | 0.25 | KOC(CH₃)₃ | 16 | 26 |
| 9^{[c]} | 0.25 | K₃PO₄ | 16 | 22 |
| 10 | 0.5 | Cs₂CO₃ | 16 | 95 |
| 11 | 0.5 | Cs₂CO₃ | 2 | 24 |
| 12 | 0.5 | Cs₂CO₃ | 4 | 44 |
| 13 | 0.5 | Cs₂CO₃ | 6 | 61 |
| 14 | 0.5 | K₃PO₄ | 16 | 55 |
| 15 | 1 | K₃PO₄ | 16 | 57 |
| 16 | 2 | K₃PO₄ | 16 | 56 |
| 17^{[d]} | 1 | K₃PO₄ | 16 | 96 (85) |
| 18^{[d]} | 0.75 | K₃PO₄ | 16 | 86 |
| 19^{[d]} | 0.5 | K₃PO₄ | 16 | 78 |
| 20^{[d]} | 0.25 | K₃PO₄ | 16 | 33 |

General conditions: iodobenzene (715 μL, 6.42 mmol, 1.5 equiv), pyrrole (300 μL, 4.29 mmol, 1 equiv), base (2 equiv) [Cu₂(μ-salben)₂] in 3.5 mL of MeCN. [^{a}] The catalyst loading refers to the amount of pyrrole.^{[b]} ¹H NMR yield, isolated yields in brackets.^{[c]} Tetrahydrofuran (THF) as solvent.^{[d]} K₃PO₄ (3.64 g, 17.18 mmol, 4 equiv).

TABLE 3
Screening of solvent and bases.

| | | | NMR |
|---|---|---|---|
| Entry | solvent | Base | yield [%] |
| 21 | DMF ^{[a]} | Cs₂CO₃ | 8 |
| 22 | DMF | K₃PO₄ | 6 |
| 23 | DMF | KOC(CH₃)₃ | 25 |
| 24 | DMSO ^{[a]} | Cs₂CO₃ | 12 |
| 25 | DMSO | K₂CO₃ | 7 |
| 26 | DMSO | NACH₃COO | 3 |
| 27 | DMSO | NaOC(CH₃)₃ | 15 |
| 28 | DMSO | KOC(CH₃)₃ | 17 |
| 29 | toluene | KOC(CH₃)₃ | 6 |
| 30 | toluene | K₃PO₄ | 2 |
| 31 | t-amyl alcohol | NaOC(CH₃)₃ | 6 |
| 32 | t-amyl alcohol | K₃PO₄ | 19 |
| 33 | THF | Cs₂CO₃ | 7 |
| 34 | THF | K₃PO₄ | 31 |
| 35 | THF | NaOC(CH₃)₃ | 9 |
| 36 | THF | KOC(CH₃)₃ | 9 |
| 37 | EtOH ^{[a]} | Cs₂CO₃ | 8 |
| 38 | EtOH | K₃PO₄ | 17 |
| 39 | EtOH | KOC(CH₃)₃ | 14 |
| 40 | MeCN | Cs₂CO₃ | 27 |
| 41 | MeCN | K₃PO₄ | 40 |

General conditions: iodobenzene (215 μL, 1.93 mmol, 1.5 equiv), pyrrole (90 μL, 1.29 mmol, 1 equiv), base (2.58 mmol, 2 equiv), [Cu₂(μ-salben)₂](2.52 mg, 0.003 mmol, 0.0025 equiv) in 1 mL of solvent; T=70-82 °C, t=16 h.^{[a]}DMF=dimethylformamide, DMSO=dimethylsulfoxide, EtOH=ethanol.

TABLE 4
Activity of the different copper (II) derivatives as catalysts.

| Entry | Base | Catalyst | Cat loading | NMR yield [%] |
|---|---|---|---|---|
| 42 | Cs₂CO₃ | [Cu₂(μ-salben)₂] | 0.25 mol % | 91 ^{[a]} |
| 43 | Cs₂CO₃ | [Cu₂(μ-sal(2- | 0.25 mol % | 52 |
| | | OMe)ben)₂] | | |
| 44 | Cs₂CO₃ | [Cu₂(μ-sal(2,6- | 0.25 mol % | 40 |
| | | diOMe)ben)₂] | | |
| 45 | Cs₂CO₃ | [Cu₂(μ-sal(4- | 0.25 mol % | 69 ^{[a]} |
| | | Cl)ben)₂] | | |
| 46 | Cs₂CO₃ | [Cu₂(μ-sal(2- | 0.25 mol % | 83 ^{[a]} |
| | | Cl)ben)₂] | | |
| 47 | Cs₂CO₃ | [Cu₂(μ-sal(4- | 0.25 mol % | 81 ^{[a]} |
| | | OMe)ben)₂] | | |
| 48 | K₃PO₄ | [Cu₂(μ-salben)₂] | 0.25 mol % | 47 |
| 49 | K₃PO₄ | [Cu₂(μ-sal(2- | 0.25 mol % | 47 |
| | | OMe)ben)₂] | | |
| 50 | K₃PO₄ | [Cu₂(μ-sal(2,6- | 0.25 mol % | 43 |
| | | diOMe)ben)₂] | | |
| 51 | Cs₂CO₃ | [Cu₂(μ-salben)₂] | 0.50 mol % | 100 |
| 52 | Cs₂CO₃ | [Cu₂(μ-sal(2- | 0.50 mol % | 94 |
| | | OMe)ben)₂] | | |
| 53 | Cs₂CO₃ | [Cu₂(μ-sal(4- | 0.50 mol % | 87 |
| | | OMe)ben)₂] | | |
| 54 | Cs₂CO₃ | [Cu₂(μ-sal(2,6- | 0.50 mol % | 95 |
| | | diOMe)ben)₂] | | |
| 55 | Cs₂CO₃ | [Cu₂(μ-sal(2- | 0.50 mol % | 89 |
| | | Cl)ben)₂] | | |
| 56 | Cs₂CO₃ | [Cu₂(μ-sal(4- | 0.50 mol % | 93 |
| | | Cl)ben)₂] | | |
| 57 | Cs₂CO₃ | [Cu₂(μ- | 0.50 mol % | 89 |
| | | sal(2)thn)₂] | | |
| 58 | Cs₂CO₃ | [Cu₂(μ- | 0.50 mol % | 96 |
| | | sal(3)thn)₂] | | |
| 59 | Cs₂CO₃ | [Cu₂(μ-sal(4- | 0.50 mol % | 99 |
| | | tBu)ben)₂] | | |
| 60 | Cs₂CO₃ | [Cu₂(μ-(5- | 0.50 mol % | 74 |
| | | Me)salben)₂] | | |
| 61 | Cs₂CO₃ | [Cu₂(μ-(5- | 0.50 mol % | 88 |
| | | OMe)salben)₂] | | |
| 62 | Cs₂CO₃ | [Cu₂(μ-(4- | 0.50 mol % | 96 |
| | | OMe)salben)₂] | | |
| 63 | Cs₂CO₃ | [Cu₂(μ-(5- | 0.50 mol % | 95 |
| | | Cl)salben)₂] | | |
| 64 | Cs₂CO₃ | [Cu₂(μ-(5- | 0.50 mol % | 88 |
| | | F)salben)₂] | | |
| 65 | Cs₂CO₃ | [Cu₂(μ-(5- | 0.50 mol % | 89 |
| | | Cl)sal(2- | | |
| | | OMe)ben)₂] | | |
| 66 | Cs₂CO₃ | [Cu₂(μ-(5- | 0.50 mol % | 69 |
| | | Cl)sal(2- | | |
| | | NO₂)ben)₂] | | |
| 67 | Cs₂CO₃ | [Cu₂(μ-(5- | 0.50 mol % | 65 |
| | | OMe)sal(2- | | |
| | | NO₂)ben)₂] | | |
| 68 | Cs₂CO₃ | [Cu₂(μ-(3- | 0.50 mol % | 98 |
| | | OMe)sal(2- | | |
| | | Cl)ben)₂] | | |
| 69 | Cs₂CO₃ | [Cu₂(μ-(5- | 0.50 mol % | 87 |
| | | OMe)sal(2- | | |
| | | Cl)ben)₂] | | |
| 70 | Cs₂CO₃ | [Cu₂(μ-(5- | 0.50 mol % | 95 |
| | | OMe)sal(2- | | |
| | | OMe)ben)₂] | | |
| 71 | Cs₂CO₃ | [Cu₂(μ-sal(2- | 0.50 mol % | 64 |
| | | NO₂)ben)₂] | | |
| 72 | Cs₂CO₃ | [Cu₂(μ- | 0.50 mol % | 93 |
| | | (salmyr)₂] | | |
| 73 | Cs₂CO₃ | [Cu₂(μ-salmen)₂] | 0.50 mol % | 92 |
| 74 | Cs₂CO₃ | [Cu₂(μ-(5- | 0.50 mol % | 97 |
| | | Me)salmen)₂] | | |
| 75 | Cs₂CO₃ | [Cu₂(μ-(5- | 0.50 mol % | 70 |
| | | OMe)salmen)₂] | | |
| 76 | Cs₂CO₃ | [Cu₂(μ-(5- | 0.50 mol % | 89 |
| | | Cl)salmen)₂] | | |

General conditions: iodobenzene (215 μL, 1.93 mmol, 1.5 equiv), pyrrole (90 μL, 1.29 mmol, 1 equiv), base (2.58 mmol, 2 equiv), 1 mL of MeCN; T=82 °C, t=16 h.^{[a]} LC/MS yield (%).

TABLE 5
Influence of various equivalents of base and iodobenzene.

| | | | NMR yield |
|---|---|---|---|
| Entry | Iodobenzene (equiv) | Base (equiv) | [%] |
| 77 | 1.5 | 2 | 45 |
| 78 | 1.2 | 2 | 35 |
| 79 | 1 | 2 | 33 |
| 80 | 1.5 | 1.1 | 28 |
| 81 | 1.2 | 1.1 | 29 |
| 82 | 1 | 1.1 | 26 |

General conditions: iodobenzene, pyrrole (90 μL, 1.29 mmol, 1 equiv), K₃PO₄, [Cu₂(μ-salben)₂] (0.006 mmol, 0.005 equiv) in 1 mL of MeCN; T=82 °C, t=16 h.

### 1.3. Substrate Scope

TABLE 6
Substrate scope with aryl iodides

| | | | Yield % |
|---|---|---|---|
| Aryl iodide | N-Nucleophile | Product | (LC/MS) |
| 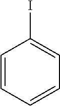 | 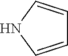 | 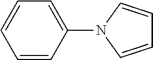 [SMILES: c1ccc(-n2cccc2)cc1] | 100 |
| 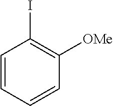 | 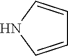 | 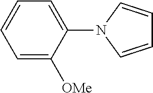 [SMILES: COc1ccccc1-n1cccc1] | 81 |
| 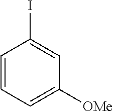 | 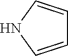 | 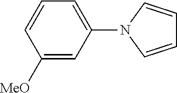 [SMILES: COc1cccc(-n2cccc2)c1] | 89 |
| 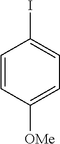 | 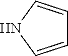 | 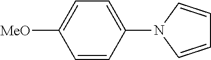 [SMILES: COc1ccc(-n2cccc2)cc1] | 98 |
| 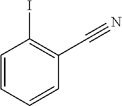 | 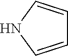 | 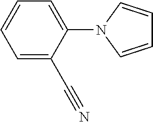 [SMILES: N#Cc1ccccc1-n1cccc1] | 93 |
| 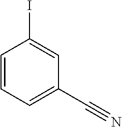 | 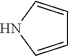 | 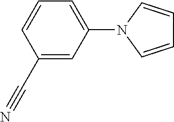 [SMILES: N#Cc1cccc(-n2cccc2)c1] | 98 |
| 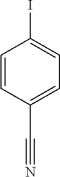 | 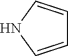 | 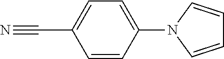 [SMILES: N#Cc1ccc(-n2cccc2)cc1] | 87 |
| 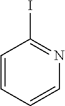 | 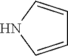 | 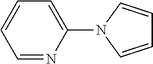 [SMILES: c1ccc(-n2cccc2)nc1] | 81 |
| 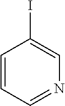 | 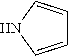 | 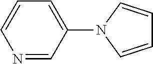 [SMILES: c1cncc(-n2cccc2)c1] | 98 |
| 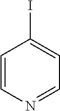 | 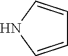 | 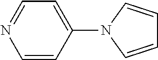 [SMILES: c1ccn(-c2ccncc2)c1] | 58 |
| 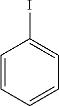 | 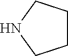 | 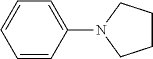 [SMILES: c1ccc(N2CCCC2)cc1] | 90 |
| 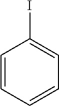 | 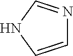 | 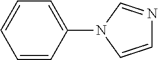 [SMILES: c1ccc(-n2ccnc2)cc1] | 89 |
| 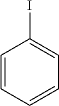 | 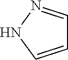 | 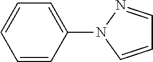 [SMILES: c1ccc(-n2cccn2)cc1] | 70 |
| 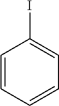 | 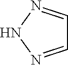 | 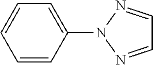 [SMILES: c1ccc(-n2nccn2)cc1] | 60 |
| 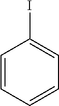 | 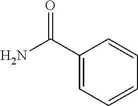 | 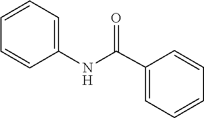 [SMILES: O=C(Nc1ccccc1)c1ccccc1] | 49 |
| 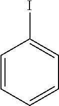 | 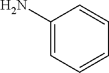 | 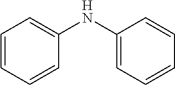 [SMILES: c1ccc(Nc2ccccc2)cc1] | 90 |
| 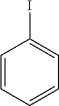 | 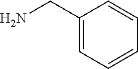 | 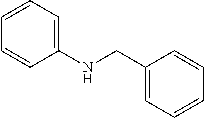 [SMILES: c1ccc(CNc2ccccc2)cc1] | 82 |
| 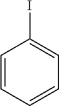 | 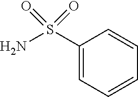 | 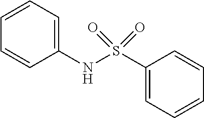 [SMILES: O=S(=O)(Nc1ccccc1)c1ccccc1] | 63 |

General conditions: aryl iodide (1.5 equiv), N-nucleophile (1 eq), CS₂CO₃ (2 equiv), [Cu₂(μ-salben)₂] (0.5 mol %) in MeCN, c=1 mol L⁻¹; T=82 °C, t=16 h.

TABLE 7
Substrate scope with aryl bromides

| | N- | | base | | yield |
|---|---|---|---|---|---|
| aryl bromide | nucleophile | product | (equiv) | solvent | (LC/MS) |
| 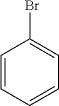 | 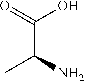 (3 equiv) | 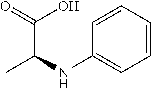 | K₃PO₄ (3 equiv) | DMSO | 66% |
| 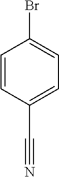 | 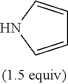 | 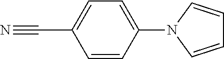 [SMILES: N#Cc1ccc(-n2cccc2)cc1] | Cs₂CO₃ (2 equiv) | MeCN | 55% |
| 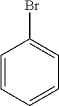 | 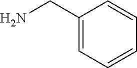 (1.5 equiv) | 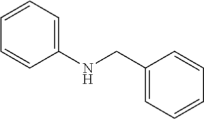 [SMILES: c1ccc(CNc2ccccc2)cc1] | Cs₂CO₃ (2 equiv) | MeCN | 51% |
| 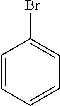 | 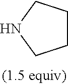 | 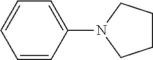 [SMILES: c1ccc(N2CCCC2)cc1] | Cs₂CO₃ (2 equiv) | MeCN | 20% |
| 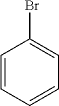 | 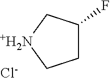 (1.5 equiv) | 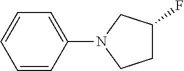 [SMILES: F[C@@H]1CCN(c2ccccc2)C1] | Cs₂CO₃ (2 equiv) | MeCN | 48% |
| 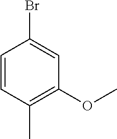 | 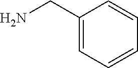 (1.5 equiv) | 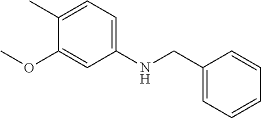 [SMILES: COc1cc(NCc2ccccc2)ccc1C] | Cs₂CO₃ (2 equiv) | MeCN | 54% |
| 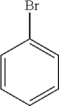 | 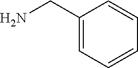 (1.5 equiv) | 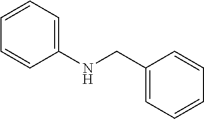 [SMILES: c1ccc(CNc2ccccc2)cc1] | K₃PO₄ (3 equiv) | DMSO | 63% |
| 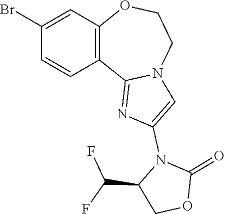 [SMILES: O=C1OC[C@@H](C(F)F)N1c1cn2c(n1)-c1ccc(Br)cc1OCC2] | 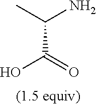 | 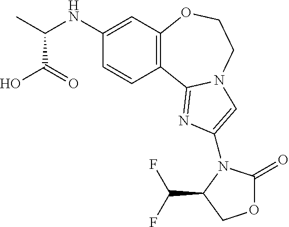 [SMILES: C[C@H](Nc1ccc2c(c1)OCCn1cc(N3C(=O)OC[C@H]3C(F)F)nc1-2)C(=O)O] | K₃PO₄ (3 equiv) t = 20 h | DMSO | 94% |

General conditions: aryl bromide (1 equiv), N-nuclephile (n equiv as reported in the table), base, [Cu₂(μ-salben)₂](2.5 mol %), base (n equiv as reported in the table), solvent, c=0.5 mol L⁻¹; T=85 °C, t=16 h.

### 2. Synthesis of the Novel Salben-Type Ligands

### 2.1 Synthesis of H₂sal(2,6-diOMe)ben

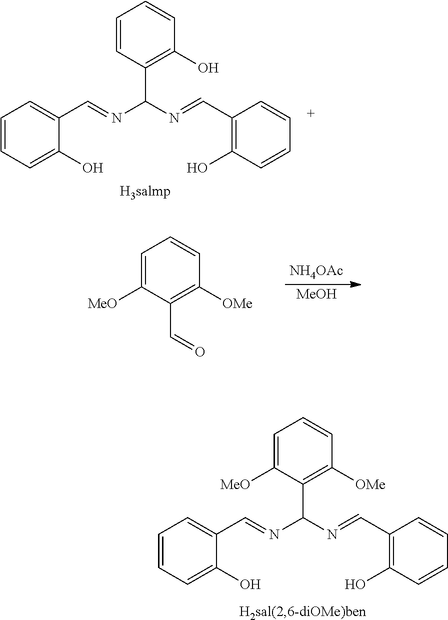

H₃salmp (0.499 g, 1.44 mmol) and 2,6-dimethoxybenzaldehyde (0.478 g, 2.87 mmol) were added to a solution of ammonium acetate (0.113 g, 1.46 mmol) in 5 mL of methanol (MeOH), obtaining a yellow suspension that was left under magnetic stirring for 24 h at room temperature (RT). The yellow product was then recovered by filtration, washed with MeOH (2×2 mL) and diethyl ether (Et₂O) (2×2 mL) and dried under vacuum for several hours, yielding 0.645 g of a bright yellow powdery solid (76% yield). Elemental analysis for C₂₃H₂₂N₂O₄, MW=390.44 g/mol, calculated: C, 70.75%; H 5.68%; N 7.17%. Found: C, 70.54%; H, 5.79%, N, 7.24%. IR spectrum, cm⁻¹ (ATR): 3050-2830, 1628, 1585, 1493-1474, 1397, 1276, 1250, 1110-1067, 758. ¹H NMR, 298 K, CDCl₃, 400 MHz: δ (ppm) 13.62 (2H, s), 8.55 (2H, d), 7.32-7.28 (4H, m), 7.26 (1H, t), 6.95 (2H, dd), 6.87 (2H, td), 6.77 (1H, s), 6.58 (2H, d), 3.85 (3H, s). ¹³C NMR, 298 K, CDCl₃, 101 MHz: δ (ppm) 163.4, 161.5, 158.5, 132.6, 132.0, 130.3, 119.1, 118.6, 117.3, 115.6, 104.7, 79.6, 56.0.

### 2.2 Synthesis of H₂sal(2)thn

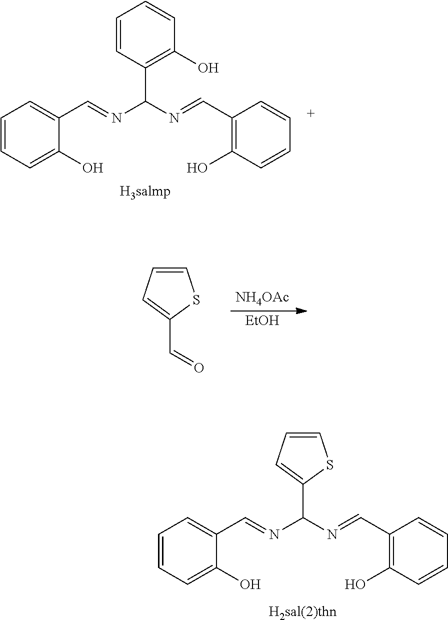

H₃salmp (3.012 g, 8.695 mmol) was added to a solution of ammonium acetate (2.024 g, 26.26 mmol) in EtOH (50 mL). Thiophene-2-carboxaldehyde (1.70 mL, 18.5 mmol) was then added to the suspension, and the reaction mixture was stirred at RT for 4 days. The formed pale-yellow solid was filtered, washed with EtOH and diisopropyl ether (iPr₂O) and dried under vacuum for several hours, yielding 2.946 g of the title compound (67%). Elemental analysis for C₁₉H₁₆N₂O₂S, MW=336.41, calculated: C, 67.84%; H, 4.79%; N, 8.33%; S 9.53. Found: C, 67.77%; H, 4.84%; N, 8.15%; S 10.05%. m.p.: 109-112 °C. IR spectrum, cm⁻¹ (ATR): 3109, 2853, 1618, 1575, 1497, 1456, 1422, 1372, 1357, 1272, 1040, 1025, 975, 897, 753, 712, 598. ¹H NMR, 298 K, acetone-d₆, 400 MHz: δ (ppm) 6.44 (s, 1H), 6.94 (dd, 2H), 7.08 (dd, 2H), 7.24 (dt, 2H), 7.40 (ddd, 2H), 7.51 (dd, 2H), 7.52 (dd, 2H), 8.85 (s, 2H), 12.82 (s, 2H). ¹³C NMR, 298 K, acetone-d₆, 101 MHz: δ (ppm) 86.2, 116.8, 118.7, 119.0, 125.2, 126.8, 127.2, 132.8, 133.4, 144.4, 161.1, 166.5. Mass spectrum (ESI⁺) in MeOH, m/z (intensity %): calculated for [M+H]⁺: 337.1011; found: 337.1000 (100).

### 2.3 Synthesis of H₂sal(3)thn

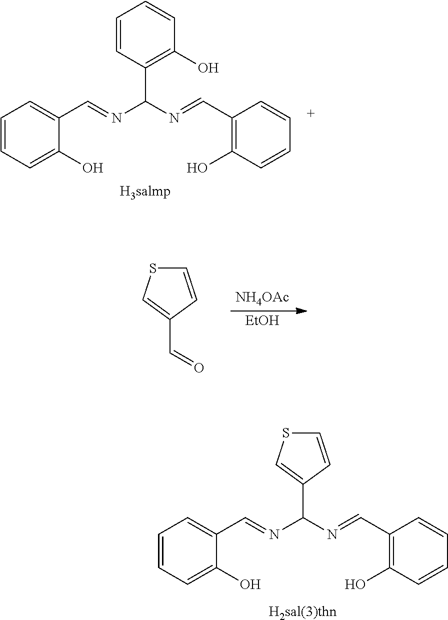

H₃salmp (3.011 g, 8.694 mmol) was added to a solution of ammonium acetate (2.017 g, 26.17 mmol) in EtOH (30 mL). Thiophene-3-carboxaldehyde (2.012 g, 17.94 mmol) was added to the suspension, and the reaction mixture was stirred at RT for 4 days. The formed pale-yellow solid was filtered, washed with EtOH and iPr₂O and dried under vacuum, yielding 3.268 g of the title compound (75%). Elemental analysis for C₁₉H₁₆N₂O₂S, MW=336.41, calculated: C, 67.84%, H, 4.79%, N, 8.33%, S 9.53%. Found: C, 67.18%, H, 4.76%, N, 8.55%, S 10.50%. m.p.: 94-96 °C. IR spectrum, cm⁻¹ (ATR): 3107, 3054, 3000, 2858, 1618, 1576, 1500, 1456, 1424, 1368, 1273, 1043, 976, 779, 752, 733, 635. ¹H NMR, 298 K, acetone-d₆, 400 MHz: δ (ppm) 6.26 (s, 1H), 6.94 (d, 2H), 6.96 (dd, 2H), 7.29 (dd, 2H), 7.39 (dd, 2H), 7.50 (dd, 2H), 7.54 (dd, 1H), 7.56 (m, 1H), 8.83 (s, 2H), 13.01 (s, 2H). ¹³C NMR, 298 K, acetone-d₆, 101 MHz: δ (ppm) 87.5, 117.6, 119.6, 119.7, 123.4, 127.0, 127.8, 133.5, 133.9, 143.0, 162.0, 167.0. Mass spectrum (ESI⁺) in MeOH, m/z (intensity %): calculated for [M+H]⁺: 337.1011; found: 337.1001 (100).

### 2.4.1 Synthesis of (R)—H₂salmyr

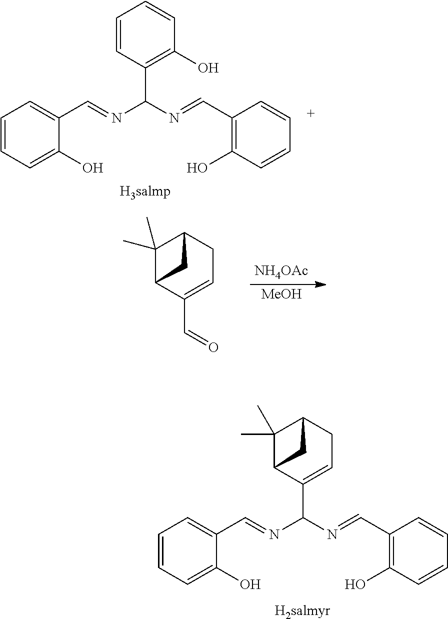

H₃salmp (0.30135 g, 0.870 mmol) was added to a solution of NH₄OAc (0.20471 g, 2.66 mmol) in MeOH (10 mL). (R)-Myrtenal (0.25308 g, 1.68 mmol) was added to the yellow suspension, and it was stirred at RT for 1 d, during which H₃salmp completely dissolved in 6 h and the desired product precipitated after 12 h. The formed pale-yellow solid was filtered, washed with MeOH (3×3 mL) and iPr₂O (3×3 mL) and dried under vacuum for 1 h. Yield: 0.29610 g (61%). Elemental analysis calcd (%) for C₂₄H₂₆N₂O₂ (374.48): C, 76.98, H, 7.00, N, 7.48. Found: C, 76.78, H, 7.04, N, 7.51. Melting point: 184-187 °C. ¹H NMR (400 MHz, 298 K, acetone-d₆) in ppm: δ 0.86 (3H, s), 1.43 (3H, s), 1.85 (1H, d, 10 Hz), 2.00 (1H, dd, 2-4 Hz), 2.14-2.19 (1H, m), 2.57-2.66 (2H, m), 3.45 (1H, t, 5 Hz), 4.40 (1H, d, 8 Hz), 6.83 (1H, s), 6.86-6.94 (4H, m, 1-7 Hz), 7.30-7.34 (2H, m, 2 Hz), 7.37 (1H, dd, 2-8 Hz), 7.43 (1H, dd, 2-8 Hz), 8.40 (1H, s), 8.61 (1, s), 13.15 (1H, s), 13.20 (1H, s). ¹³C NMR (101 MHz, 298 K acetone-d₆) in ppm: δ 22.3, 26.4, 27.5, 34.9, 41.5, 42.0, 45.9, 64.2, 117.4, 117.4, 119.5, 119.8, 120.1, 120.4, 132.6, 132.7, 133.0, 133.3, 138.5, 144.8, 161.6, 161.9, 163.3, 164.2. IR-ATR (cm⁻¹): 2993, 2969, 2949, 2865, 1645, 1620, 1596, 1563, 1495, 1456, 1413, 1300, 1278, 1244, 1148, 1079, 971, 844, 753. Mass spectrum (ESI⁺) in MeCN, m/z (intensity %): calculated for [M+H]+: 375.2073; found: 375.2060 (100).

### 2.4.2 Synthesis of (R)—H₂salC1myr

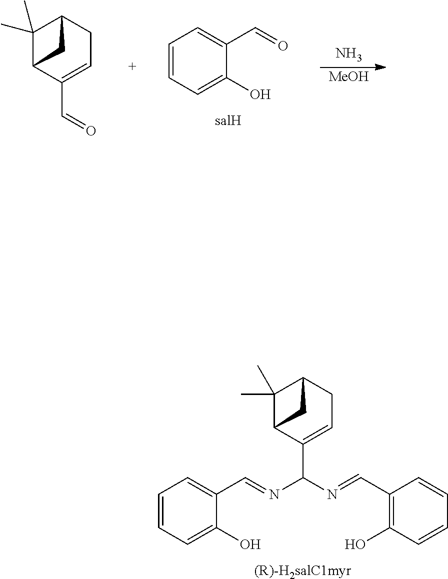

(R)-Myrtenal (0.1352 g, 0.900 mmol) was added to a solution of NH₃ (750 μL of an aqueous solution 25% m/m, 10.0 mmol) in MeOH (2.5 mL). Salicylaldehyde (salH) (0.2001 g, 1.64 mmol) was added to the colourless solution, and the mixture was stirred at RT for 3 h with the formation of the desired product as precipitate. The formed pale-yellow solid was filtered, washed with MeOH (3×3 mL) and iPr₂O (3×3 mL) and dried under vacuum overnight. Yield: 0.2112 g (69%). Elemental analysis calcd (%) for C₂₄H₂₆N₂O₂·0.2 H₂O (382.08): C, 76.24, H, 7.04, N, 7.41. Found: C, 76.04, H, 6.68, N, 7.38. Melting point: 135-139 °C. ¹H NMR (400 MHz, 298 K, acetone-d₆) in ppm: δ 0.79 (3H, s), 1.28 (3H, s), 1.85 (1H, d, 10 Hz), 2.09-2.14 (1H, m), 2.27-2.42 (2H, m, 6 Hz), 2.45-2.48 (2H, m), 5.49 (1H, s), 5.76 (1H, s), 6.91-6.95 (4H, m), 7.35-7.40 (2H, m), 7.48 (2H, dd, 1-8 Hz), 8.71 (1H, s), 8.73 (1H, s), 13.04 (1H, s), 13.06 (1H, s). ¹³C NMR (101 MHz, 298 K acetone-d₆) in ppm: δ 21.6, 26.4, 32.0, 32.3, 38.6, 41.6, 43.1, 91.7, 117.6, 119.6, 121.4, 121.4, 133.3, 133.7, 148.2, 162.0, 166.6, 166.8. IR-ATR (cm⁻¹): 2989, 2969, 2927, 2877, 1620, 1574, 1494, 1457, 1413, 1365, 1274, 1212, 1150, 1039, 976, 897, 836, 751. Mass spectrum (ESI⁺) in MeCN, m/z (intensity %): calculated for [M+H]+: 375.2073; found: 335.2067 (100.

### 2.6 Synthesis of H₂(4-OMe)salben

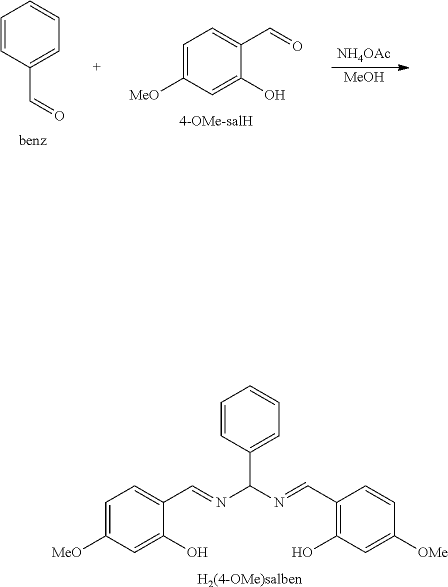

Benzaldehyde (0.1631 mg, 1.537 mmol) was added to a supersaturated solution of ammonium acetate (0.1631 g, 1.5368 mmol) in MeOH (2.5 mL) and stirred at RT for 5 min under inert atmosphere. 4-OMe-salH (0.4175 g, 2.734 mmol) was then added to the colourless solution, and the reaction mixture was stirred at RT for 4 h. The formed pale-grey solid was filtered, washed with MeOH (3×3 mL) and dried under vacuum for 1 h. Yield 0.2615 g (49%). Elemental analysis calcd (%) for C₂₃H₂₂N₂O₄, (390.44): C, 70.76, H, 5.68, N, 7.16. Found: C, 70.70, H, 5.80, N, 6.85. Melting Point: 111-112 °C. ¹H NMR (400 MHz, 298 K, acetone-d₆) in ppm: δ 13.44 (2H, s), 8.74 (2H, s), 7.56 (2H, d, 8 Hz), 7.47-7.43 (2H, m), 7.40 (2H, d, 8 Hz), 7.39-7.34 (1H, m), 6.51 (2H, dd, 8-2 Hz), 6.46 (2H, d, 2 Hz), 6.03 (1H, s), 3.84 (6H, s). ¹³C NMR (151 MHz, 298 K, acetone-d₆) δ 164.9, 163.9, 163.5, 141.8, 133.8, 128.8, 128.2, 127.0, 112.6, 106.4, 100.8, 90.4, 54.9. IR-ATR (cm⁻¹): 1610, 1570, 1452, 1316, 1288, 1240, 1168, 764. Mass spectrum (ESI⁺) in MeCN, m z (intensity %): calculated for [M+H]+: 391.1658; found: 391.1639 (100).

### 2.7 Synthesis of H₂(5-OMe)salben

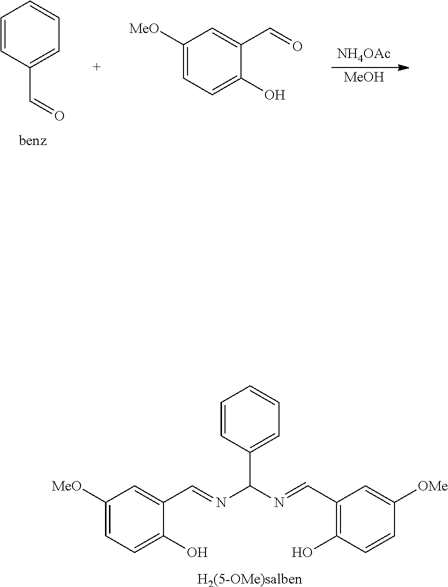 [SMILES: "MeOH" ; O=Cc1ccccc1 ; COc1ccc(O)c(C=O)c1 ; COc1ccc(O)c(/C=N/C(/N=C/c2cc(OC)ccc2O)c2ccccc2)c1 ; "NH4OAc" ; "H2(5-OMe)salben"]

Benzaldehyde (84.2 mg, 0.794 mmol) was added to a supersaturated solution of ammonium acetate (0.9672 g, 15.81 mmol) in MeOH (2.5 mL) and stirred at RT for 5 min under inert atmosphere. 5-OMe-salH (201.3 mg, 1.332 mmol) was added to the colourless solution, and the reaction mixture was stirred at RT for 3 h. The formed pale-yellow solid was filtered, washed with MeOH (3×3 mL) and iPr₂O (3×3 mL) and dried under vacuum for 1 h. Yield 0.1754 g (68%). Elemental analysis calcd (%) for C₂₃H₂₂N₂O₄, (390.44): C, 70.76, H, 5.68, N, 7.16. Found: C, 71.04, H, 5.71, N, 7.26. Melting Point: 137-141 °C. ¹H NMR (600 MHz, 298 K, acetone-d₆) in ppm: δ 12.49 (2H, s), 8.82 (2H, s), 7.59-7.54 (2H, m), 7.47-7.42 (2H, m), 7.39-7.33 (1H, m), 7.08 (2H, d, 3 Hz), 7.00 (2H, dd, 9-3 Hz), 6.86 (2H, d, 9 Hz), 6.14 (1H, s), 3.76 (6H, s). ¹³C NMR (151 MHz, 298 K, acetone-d₆) δ 166.7, 156.1, 153.3, 129.8, 129.3, 127.8, 121.2, 119.3, 118.4, 116.3, 91.5, 56.1. Mass spectrum (ESI⁺) in MeCN, m/z (intensity %): calculated for [M+H]+: 391.1658; found: 391.1655 (100).

### 2.8 Synthesis of H₂(5-F)salben

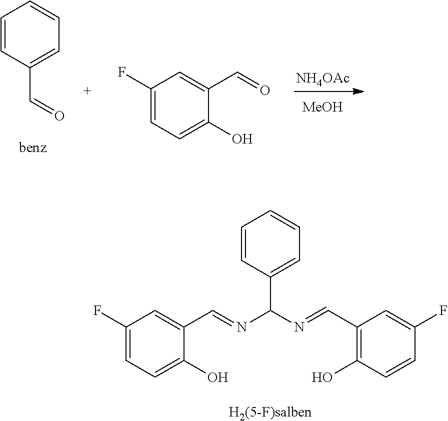 [SMILES: "MeOH" ; O=Cc1ccccc1 ; O=Cc1cc(F)ccc1O ; Oc1ccc(F)cc1/C=N/C(/N=C/c1cc(F)ccc1O)c1ccccc1 ; "NH4OAc"]

Benzaldehyde (83.2 mg, 0.784 mmol) was added to a supersaturated solution of ammonium acetate (0.8828 g, 11.442 mmol) in MeOH (2.5 mL) and stirred at RT for 5 min. 5-F-salH (206.1 mg, 1.4708 mmol) was added to the colourless solution, and the reaction mixture was stirred at RT for 3 h. The formed yellow solid was filtered, washed with MeOH (3×3 mL) and iPr₂O (3×3 mL) and dried under vacuum for 1 h. Yield 0.2295 g (85%). Elemental analysis calcd (%) for C₂₁H₁₆F₂N₂O₂, (366.12): C, 68.45, H, 4.40, N, 7.65. Found: C, 68.19, H, 4.32, N, 7.54. Melting Point: 144-147 °C. ¹H NMR (600 MHz, 298 K, acetone-d₆) δ 12.78 (2H, d, 4 Hz), 8.86 (2H, d, 3 Hz), 7.58 (2H, d, 8 Hz), 7.46 (2H, dd, 8 Hz), 7.43-7.35 (1H, m), 7.32 (2H, ddd, 9-3-2 Hz), 7.19 (2H, ddd, 9-3 Hz), 6.94 (2H, ddd, 9-4-2 Hz), 6.20 (2 Hz). ¹³C NMR (151 MHz, 298 K, acetone-d₆) δ 166.1, 158.3, 156.3 (d, J_{C-F}=233 Hz), 141.7, 129.8, 129.4, 127.9, 120.8 (d, 23 Hz), 119.6 (d, 8 Hz) 118.9 (d, 8 Hz), 118.3 (d, 23 Hz), 91.4. Mass spectrum (ESI⁺) in MeCN, m z (intensity %): calculated for [M+H]: 367.1258; found: 367.1254 (100).

### 2.9 Synthesis of H₂(5-Cl)salben

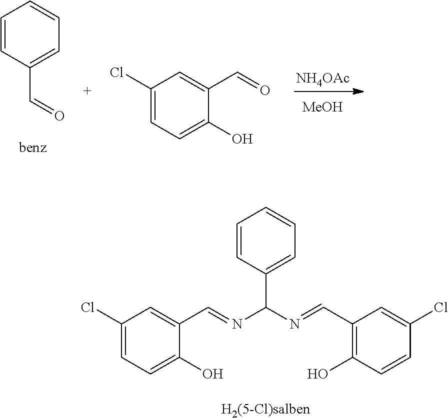 [SMILES: "MeOH" ; O=Cc1ccccc1 ; O=Cc1cc(Cl)ccc1O ; Oc1ccc(Cl)cc1/C=N/C(/N=C/c1cc(Cl)ccc1O)c1ccccc1 ; "NH4OAc"]

Benzaldehyde (81.1 mg, 0.764 mmol) was added to a supersaturated solution of ammonium acetate (0.9869 g, 12.80 mmol) in MeOH (3 mL) and stirred at RT for 5 min. 5-Cl-salH (200.6 mg, 1.281 mmol) was added to the colourless solution, and the reaction mixture was stirred at RT for 3 h. The formed yellow solid was filtered, washed with MeOH (3×3 mL) and iPr₂O (3×3 mL) and dried under vacuum for 1 h. Yield 0.1567 g (61%). Elemental analysis calcd (%) for C₂₁H₁₆Cl₂N₂O₂ 0.3 H₂O (405.28): C, 62.33, H, 4.13, N, 6.92. Found: C, 62.33, H, 3.96, N, 6.82. Melting Point: 128-131 °C. ¹H NMR (600 MHz, 298 K acetone-d₆) δ 13.05 (2H, s), 8.86 (2H, s), 7.60-7.54 (4H, m, 2-7 Hz), 7.49-7.40 (2H, m(dd), 7 Hz), 7.40-7.34 (3H, m, 2-9 Hz), 6.95 (2H, d, 9 Hz), 6.21 (1H, s). ¹³C NMR (151 MHz, 298 K, acetone-d₆) δ 166.1, 160.7, 141.6, 133.6, 132.5, 129.9, 129.5, 127.9, 123.8, 120.8, 119.5, 91.2. Mass spectrum (ESI⁺) in MeCN, m/z (intensity %): calculated for [M+Na]+: 399.0667; found: 399.0661 (100).

### 2.10 Synthesis of H₂(5-Me)salben

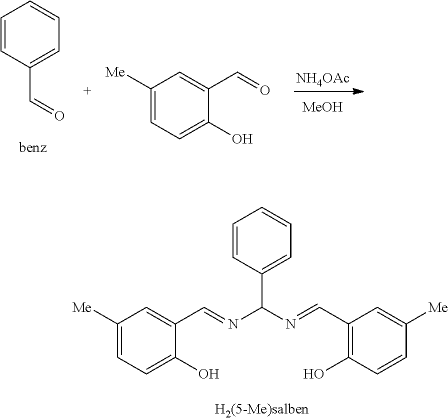 [SMILES: "MeOH" ; O=Cc1ccccc1 ; Cc1ccc(O)c(C=O)c1 ; Cc1ccc(O)c(/C=N/C(/N=C/c2cc(C)ccc2O)c2ccccc2)c1 ; "NH4OAc"]

Benzaldehyde (84.2 mg, 0.8015 mmol) was added to a supersaturated solution of ammonium acetate (0.8636 g, 11.20 mmol) in MeOH (2.5 mL) and stirred at RT for 5 min. 5-Me-salH (214.5 mg, 1.5755 mmol) was added to the colourless solution, and the reaction mixture was stirred at RT for 3 h. The formed yellow solid was filtered, washed with MeOH (3×3 mL) and dried under vacuum for 1 h. Yield 0.2041 g (72%). Elemental analysis calcd (%) for C₂₃H₂₂N₂O₂·0.05 NH₄OAc (362.29): C, 76.58, H, 6.22, N, 7.93. Found: C, 76.38, H, 5.98, N, 7.80.

Melting Point: 125-130 °C. ¹H NMR (400 MHz, 298 K, CDCl₃) δ 12.72 (2H, s), 8.53 (2H, s), 7.49-7.29 (5H, m), 7.16 (2H, dd, 8-2 Hz), 7.11 (2H, d, 2 Hz), 6.89 (2H, d, 8 Hz), 5.99 (1H, s), 2.29 (6H, s). Mass spectrum (ESI⁺) in MeCN, m/z (intensity %): calculated for [M+H]+: 359.1758; found: 359.1747 (100).

### 2.11 Synthesis of H₂(3-OMe)sal(2-Cl)ben

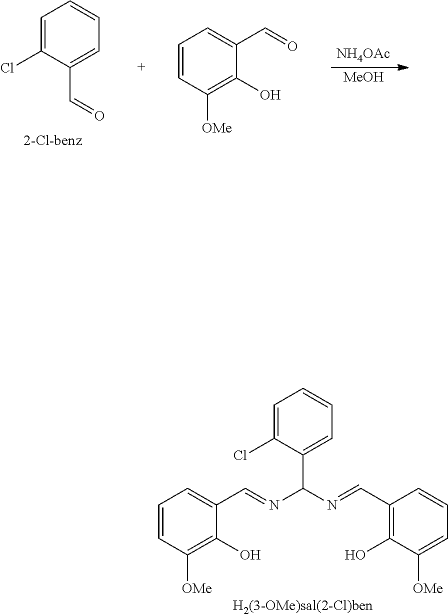 [SMILES: "MeOH" ; O=Cc1ccccc1Cl ; COc1cccc(C=O)c1O ; COc1cccc(/C=N/C(/N=C/c2cccc(OC)c2O)c2ccccc2Cl)c1O ; "NH4OAc" ; "H2(3-OMe)sal(2-Cl)ben"]

2-Cl-benzaldehyde (92.2 mg, 0.657 mmol) was added to a solution of ammonium acetate (0.8808 g, 11.40 mmol) in MeOH (10 mL) and stirred at RT for 5 min. 3-OMe-salH (0.2085 g, 1.370 mmol) was added to the colourless solution, and the reaction mixture was stirred at RT for 3 h. The formed yellow solid was filtered, washed with MeOH (3×3 mL) and dried under vacuum for 1 h. Yield 0.1431 g (51%). Elemental analysis calcd (%) for C₂₃H₂₁ClN₂O₄(424.12): C, 65.02, H, 4.98, N, 6.59. Found: C, 64.56, H, 4.91, N, 6.54. ¹H NMR (400 MHz, 298 K, acetone-d₆) δ 13.00 (2H, s), 8.91 (2H, s), 7.72 (1H, dd, 1-8 Hz), 7.53 (1H, dd, 1-8 Hz), 7.49 (1H, dd, 1-7 Hz), 7.43 (1H, dd, 2-7H) 7.15 (2H, d, 8 Hz), 7.11 (2H, d, 8 Hz), 6.89 (2H, dd, 8 Hz), 6.55 (1H, s), 3.85 (6H, s). ¹³C NMR (101 MHz, 298 K, acetone-d₆) δ 166.7, 151.5, 148.5, 138.1, 132.6, 130.0, 129.2, 127.2, 124.1, 118.8, 118.6, 118.5, 115.8, 86.3, 55.6. IR-ATR (cm⁻¹): 1613, 1600, 1459, 1317, 1252, 1171, 764, 728. Mass spectrum (ESI⁺) in MeCN, m/z (intensity %): calculated for [M+H]+: 425.1268; found: 425.1248 (60).

### 2.12 Synthesis of H₂(5-OMe)sal(2-Cl)ben

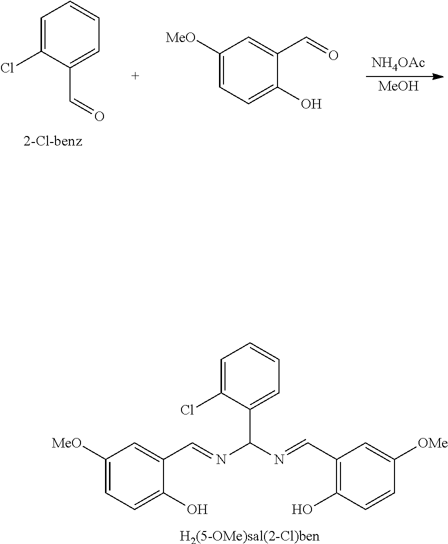 [SMILES: "MeOH" ; O=Cc1ccccc1Cl ; COc1ccc(O)c(C=O)c1 ; COc1ccc(O)c(/C=N/C(/N=C/c2cc(OC)ccc2O)c2ccccc2Cl)c1 ; "NH4OAc" ; "H2(5-OMe)sal(2-Cl)ben"]

2-Cl-benzaldehyde (0.1322 g, 0.8689 mmol) was added to a solution of ammonium acetate (0.21665 g, 2.038 mmol) in MeOH (10 mL) and stirred at RT for 5 min. 5-OMe-salH (0.2864 g, 2.038 mmol) was added to the colourless solution, and the reaction mixture was stirred at RT for 3 h. The formed yellow solid was filtered, washed with MeOH (3×3 mL) and dried under vacuum for 1 h. Yield 0.1278 g (32%). Elemental analysis calcd (%) for C₂₃H₂₁ClN₂O₄(424.12): C, 65.02, H, 4.98, N, 6.59. Found: C, 64.78, H, 4.95, N, 6.51. ¹H NMR (400 MHz, 298 K, acetone-d₆) δ 12.34 (2H, s), 8.85 (2H, s), 7.71 (1H, dd, 8-2 Hz), 7.51 (1H, dd, 8-2 Hz), 7.47 (1H, dd, 8-2 Hz), 7.41 (1H, dd, 8-2 Hz), 7.12 (2H, d, 3 Hz), 7.02 (2H, dd, 9-3 Hz), 6.86 (2H, d, 9 Hz), 6.54 (1H, s), 3.76 (6H, s). ¹³C NMR (101 MHz, 298 K, acetone-d₆) δ 166.7, 155.2, 152.5, 138.1, 132.6, 130.0, 129.2, 127.9, 120.6, 118.4, 117.6, 115.5, 115.4, 86.5, 55.2. IR-ATR (cm⁻¹): 1634, 1584, 1487, 1329, 1266, 1225, 1165, 770. Mass spectrum (ESI⁺) in MeCN, m/z (intensity %): calculated for [M+H]+: 425.1268; found: 425.1248 (60).

### 2.13 Synthesis of H₂(5-OMe)sal(2-OMe)ben

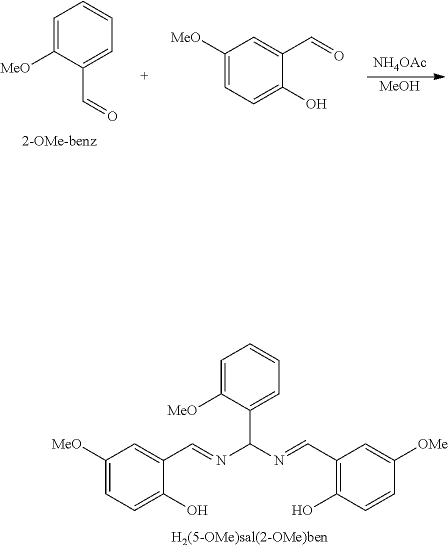 [SMILES: "MeOH" ; COc1ccccc1C=O ; COc1ccc(O)c(C=O)c1 ; COc1ccc(O)c(/C=N/C(/N=C/c2cc(OC)ccc2O)c2ccccc2OC)c1 ; "NH4OAc" ; "H2(5-OMe)sal(2-OMe)ben"]

2-OMe-benzaldehyde (0.1349 g, 0.9584 mmol) was added to a solution of ammonium acetate (0.2294 g, 2.977 mmol) in MeOH (10 mL) and stirred at RT for 5 min. 5-OMe-salH (0.2883 g, 1.895 mmol) was added to the colourless solution, and the reaction mixture was stirred at RT for 3 h. The formed yellow solid was filtered, washed with MeOH (3×3 mL) and dried under vacuum for 1 h. Yield 0.3028 g (76%). Elemental analysis calcd (%) for C₂₄H₂₄N₂O₅ (420.47): C, 68.75, H, 5.75, N, 6.62. Found: C, 69.07, H, 5.83, N, 6.68. ¹H NMR (400 MHz, 298 K, acetone-d₆) δ 12.60 (2H, s), 8.78 (2H, s), 7.49 (1H, dd, 8-2 Hz), 7.35 (1H, dd, 9 Hz), 7.10-7.07 (3H, m), 7.03 (1H, dd, 6 Hz), 7.00 (2H, dd, 9-3 Hz), 6.85 (2H, d, 9 Hz), 6.50 (1H, s), 3.96 (3H, s) 3.76 (6H, s). ¹³C NMR (101 MHz, 298 K, acetone-d₆) δ 165.7, 156.8, 154.9, 153.2, 152.8, 129.6, 128.6, 127.5, 120.8, 120.0, 118.6, 117.5, 115.4, 111.4, 84.2, 55.2. IR-ATR (cm⁻¹): 1635, 1585, 1489, 1328, 1266, 1240, 1167, 760. Mass spectrum (ESI⁺) in MeCN, m/z (intensity %): calculated for [M+H]⁺: 421.1764; found: 421.2400 (40).

### 2.14 Synthesis of H₂(5-Cl)sal(2-OMe)ben

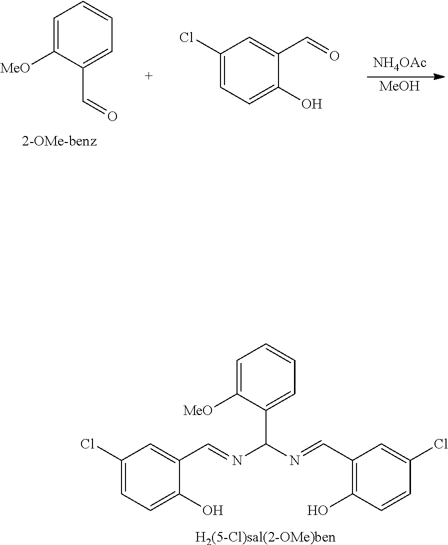 [SMILES: "MeOH" ; COc1ccccc1C=O ; O=Cc1cc(Cl)ccc1O ; COc1ccccc1C(/N=C/c1cc(Cl)ccc1O)/N=C/c1cc(Cl)ccc1O ; "NH4OAc" ; "H2(5-Cl)sal(2-OMe)ben"]

2-OMe-benzaldehyde (0.1287 g, 0.9449 mmol) was added to a solution of ammonium acetate (0.2173 g, 2.819 mmol) in MeOH (10 mL) and stirred at RT for 5 min. 5-Cl-salH (0.2918 g, 1.864 mmol) was added to the colourless solution, and the reaction mixture was stirred at RT for 3 h. The formed yellow solid was filtered, washed with MeOH (3×3 mL) and dried under vacuum for 1 h. Yield 0.2996 g (76%). Elemental analysis calcd (%) for C₂₂H₁₈Cl₂N₂O₃ (429.30): C, 61.35, H, 4.23, N, 6.53. Found: C, 61.05, H, 4.26, N, 6.55. Melting Point (°C): 152-154. ¹H NMR (400 MHz, 298 K, acetone-d₆) δ 13.23 (2H, s), 8.90 (2H, s), 7.63 (2H, d, 3 Hz), 7.55 (1H, dd, 8-2 Hz), 7.36-7.30 (3H, m), 7.09 (1H, d, 9 Hz), 7.05 (1H, ddd, 8-1 Hz), 6.90 (2H, d, 9 Hz), 6.50 (1H, s), 3.90 (3H, s). ¹³C NMR (101 MHz, 298 K, acetone-d₆) δ 166.9, 162.2, 157.9, 133.8, 133.4, 130.7, 129.6, 128.5, 121.8, 119.9, 119.8, 117.7, 112.3, 85.2, 56.1. IR-ATR (cm¹): 1606, 1521, 1453, 1384, 1297, 1242, 1165, 748. Mass spectrum (ESI⁺) in MeCN, m/z (intensity %): calculated for [M+H]+: 429.0773; found: 429.0660 (40).

### 2.15 Synthesis of H₂(5-OMe)sal(2-NO₂)ben

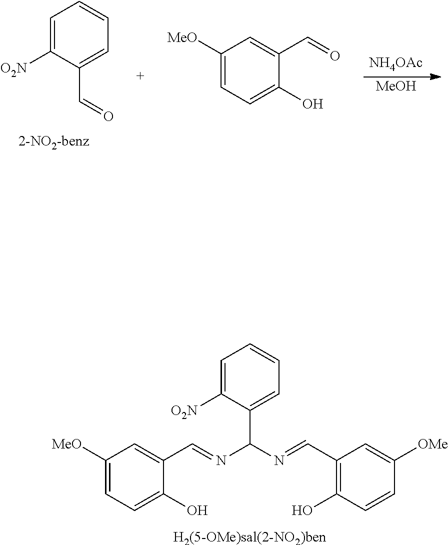 [SMILES: "MeOH" ; O=Cc1ccccc1[N+](=O)[O-] ; COc1ccc(O)c(C=O)c1 ; COc1ccc(O)c(/C=N/C(/N=C/c2cc(OC)ccc2O)c2ccccc2[N+](=O)[O-])c1 ; "NH4OAc" ; "H2(5-OMe)sal(2-NO2)ben"]

2-NO₂-benzaldehyde (0.1390 g, 0.9199 mmol) was added to a solution of ammonium acetate (0.2212 g, 2.869 mmol) in MeOH (10 mL) and stirred at RT for 5 min. 5-OMe-salH (0.2796 g, 1.837 mmol) was added to the colourless solution, and the reaction mixture was stirred at RT for 3 h. The formed yellow solid was filtered, washed with MeOH (3×3 mL) and dried under vacuum for 1 h. Yield 0.1957 g (49%). Elemental analysis calcd (%) for C₂₃H₂₁N₃O₆ (435.42): C, 63.24, H 4.86, N, 9.65. Found: C, 62.98, H, 4.90, N, 9.25. Melting Point (°C): 159-160. ¹H NMR (400 MHz, 298 K, acetone-d₆) δ 12.20 (2H, s), 8.82 (2H, s), 8.01 (1H, d, 8 Hz), 7.97 (1H, dd, 8-1 Hz), 7.86 (1H, dd, 7-1 Hz), 7.66 (1H, dd, 8-1 Hz), 7.11 (2H, d, 3 Hz), 7.01 (2H, dd, 9-3 Hz), 6.85 (2H, dd, 9 Hz), 6.66 (1H, s), 3.77 (6H, s). ¹³C NMR (101 MHz, 298 K, acetone-d₆) δ 166.9, 156.7, 154.8, 153.4, 134.7, 133.7, 129.7, 129.6, 124.5, 120.8, 118.3, 117.6, 115.6, 84.7, 55.9. IR-ATR (cm⁻¹): 1626, 1586, 1525, 1489, 1370, 1338, 1158, 767. Mass spectrum (ESI⁺) in MeCN, m/z (intensity %): calculated for [M+H]+: 436.1483; found: 436.1493 (60).

### 2.16 Synthesis of H₂(5-Cl)sal(2-NO₂)ben

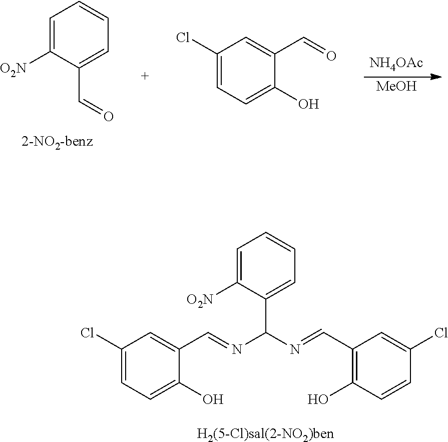 [SMILES: "MeOH" ; O=Cc1ccccc1[N+](=O)[O-] ; O=Cc1cc(Cl)ccc1O ; O=[N+]([O-])c1ccccc1C(/N=C/c1cc(Cl)ccc1O)/N=C/c1cc(Cl)ccc1O ; "H2(5-Cl)sal(2-NO2)ben" ; "NH4OAc"]

2-NO₂-benzaldehyde (0.0976 g, 0.646 mmol) was added to a solution of ammonium acetate (0.1677 g, 2.175 mmol) in MeOH (10 mL) and stirred at RT for 5 min. 5-Cl-salH (0.2096 g, 1.339 mmol) was added to the colourless solution, and the reaction mixture was stirred at RT for 3 h. The formed yellow solid was filtered, washed with MeOH (3×3 mL) and dried under vacuum for 1 h. Yield 0.477 g (50%). Elemental analysis calcd (%) for C₂₂H₁₅Cl₂N₃O₄ (443.27): C, 56.77, H, 3.40, N, 9.46. Found: C, 57.26, H, 3.42, N, 9.61. Melting Point (°C): 148-151. ¹H NMR (400 MHz, 298 K, acetone-d₆) δ 12.73 (2H, s), 8.80 (2H, s), 8.04 (1H, dd, 8-2 Hz), 7.99 (1H, dd, 7.8-1.3 Hz), 7.87 (1H, dd, 7 Hz), 7.69 (1H, dd, 8 Hz), 7.60 (2H, d, 3 Hz), 7.40 (2H, dd, 9-3 Hz), 6.95 (2H, d, 9 Hz), 6.73 (1H, s). ¹³C NMR (101 MHz, 298 K, acetone-d₆) δ 167.7, 160.4, 150.3, 135.0, 134.9, 133.3, 132.5, 130.4, 129.6, 124.9, 124.0 120.06, 119.6, 84.5. IR-ATR (cm⁻¹): 1616, 1559, 1520, 1473, 1342, 1199, 792, 751. Mass spectrum (ESI⁺) in MeCN, m/z (intensity %): calculated for [M+H]⁺: 444.0417; found: 444.0505 (40).

### 3. Synthesis of the New Copper(II) Dinuclear Complexes

### 3.1 Synthesis of [Cu₂(μ-sal(4-tBu)ben)₂]

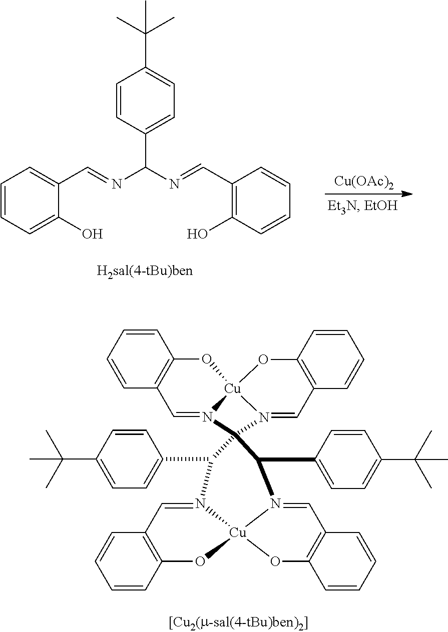

Et₃N (0.109 g, 1.08 mmol) was added to a yellow suspension of H₂sal(4-tBu)ben (0.2021 g, 0.5229 mmol) in 8 mL of EtOH. Solid Cu(OAc)₂·H₂O (0.1017 g, 0.5094 mmol) was then added obtaining a colour change to brown in a few minutes. The reaction mixture was left under magnetic stirring for 3 h at RT, and then the precipitate was isolated by filtration, washed with EtOH (2×2 mL) and dried under vacuum for several hours. Yield: 0.1882 g of a brown solid (83%). Elemental analysis calcd (%) for C₅₀H₄₈Cu₂N₄O₄·0.8EtOH (932.91): C, 66.43, H, 5.70, N, 6.01. Found: C, 66.51, H, 5.70, N, 6.01. IR-ATR (cm⁻¹): 3047, 2964, 1606, 1533, 1466, 1387, 1317, 1192, 1147, 1107, 981, 928, 827. Mass spectrum (ESI⁺) in MeCN, m/z (intensity %): calculated for [M+H]⁺: 895.2346; found: 895.2327 (100).

The ligand structure has been published in Rigamonti, L et al, Int. J. Mol. Sci. 2020, 21, 7882, DOI:: 10.3390/ijms21217882.

### 3.2 Synthesis of [Cu₂(μ-sal(2-OMe)ben)₂]

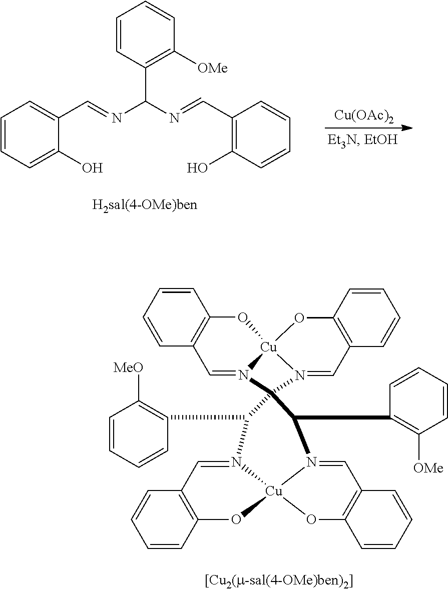

Et₃N (0.185 g, 1.83 mmol) was added to a yellow suspension of H₂sal(2-OMe)ben (0.329 g, 0.922 mmol) in 14 mL of EtOH. Copper acetate monohydrate (0.184 g, 0.912 mmol) was then added obtaining a colour change to brown/green in a few minutes. The reaction mixture was left under magnetic stirring for 3 h at RT, and then the precipitate was isolated by filtration, washed with EtOH (2×2 mL), Et₂O (2×2 mL) and dried under vacuum for several hours, yielding 0.354 g of a brown solid (92% yield). Elemental analysis for C₄₄H₃₆Cu₂N₄O₆, MW=843.9 g/mol calculated: C, 62.62%; H, 4.30%; N, 6.64%. Found: C, 61.85%; H, 4.34%, N, 6.74%. IR spectrum, cm⁻¹ (ATR): 3044-3010, 1601, 1586, 1531-1490, 1387, 1318, 1242, 1147-1026, 751. Mass spectrum (ESI⁺), m/z (intensity %): 843 [Cu₂(sal(2-OMe)ben)₂+1]⁺ (100).

### 3.3 Synthesis of [Cu₂(μ-sal(2,6-diOMe)ben)₂]

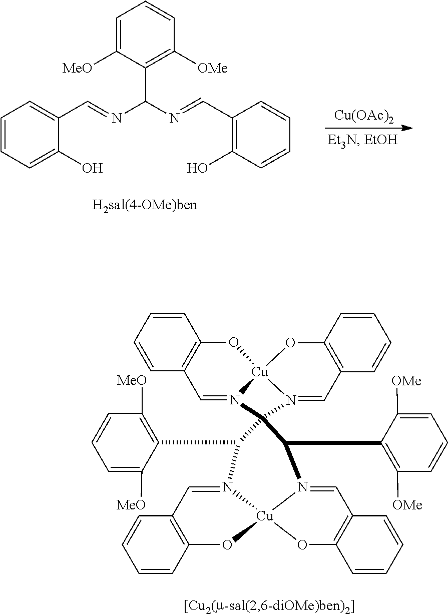

Et₃N (0.150 g, 1.48 mmol) was added to a yellow suspension of H₂sal(2,6-diOMe)ben (0.290 g, 0.743 mmol) in 7 mL of MeOH. Copper acetate monohydrate (0.148 g, 0.742 mmol) was then added obtaining a colour change to brown in a few minutes. The reaction mixture was left under magnetic stirring for 30 minutes at 0 °C, and then the precipitate was isolated by filtration, washed with cold MeOH (2×2 mL), Et₂O (2×2 mL) and dried under vacuum for several hours, yielding 0.285 g of a brown solid (85% yield). Elemental analysis for C₄₆H₄₀Cu₂N₄O₈, MW=903.94 g/mol calculated: C, 61.12%; H, 4.46%; N, 6.20%. Found: C, 60.82%; H, 4.57%, N, 6.19%. IR spectrum, cm⁻¹ (ATR): 3050-2840, 1604, 1528, 1470-1426, 1383, 1250, 1195, 1034-980, 758. Mass spectrum (ESI⁺), m/z (intensity %): 903 [Cu₂(sal(2,6-diOMe)ben)₂+1]⁺ (100).

### 3.4 Synthesis of [Cu₂(μ-sal(4-OMe)ben)₂]

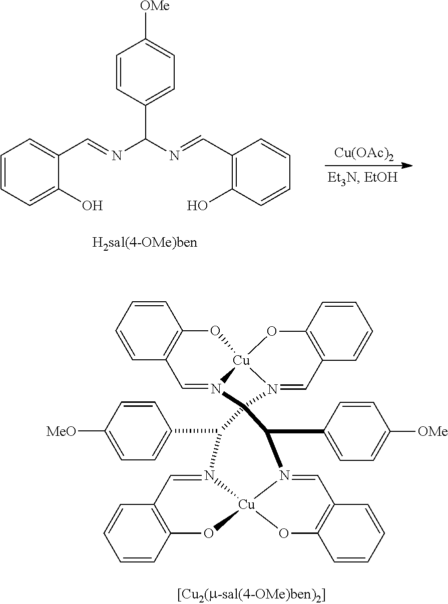

Et₃N (0.138 g, 1.36 mmol) was added to a yellow suspension of H₂sal(4-OMe)ben (0.245 g, 0.680 mmol) in 10 mL of EtOH. Copper acetate monohydrate (0.138 g, 0.690 mmol) was then added obtaining a colour change to green/brown in a few minutes. The reaction mixture was left under magnetic stirring for 3 h at RT, and then the precipitate was isolated by filtration, washed with EtOH (3 mL), iPr₂O (3 mL) and dried under vacuum for several hours, yielding 0.198 g of a brown solid (69% yield). Elemental analysis for C₄₄H₃₆Cu₂N₄O₆, MW=843.89 g/mol calculated: C, 62.62%; H, 4.30%; N, 6.64%. Found: C, 63.02%; H, 4.34%, N, 6.83%. IR spectrum, cm⁻¹ (ATR): 3050-2835, 1607, 1534, 1467-1440, 1395, 1243, 1196, 1176, 1148, 1051-979, 750, 736.

### 3.5 Synthesis of [Cu₂(μ-sal(2-Cl)ben)₂]

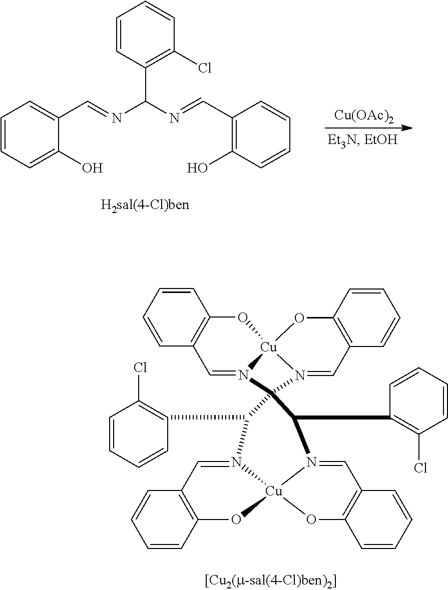

Et₃N (0.156 g, 1.54 mmol) was added to a yellow suspension of H₂sal(2-Cl)ben (0.255 g, 0.699 mmol) in 10 mL of EtOH. Copper acetate monohydrate (0.154 g, 0.774 mmol) was then added obtaining a colour change to green-brown in a few minutes. After 3 h under stirring, further 5 mL of EtOH were added and then the reaction mixture was left under magnetic stirring overnight at RT, and then the precipitate was isolated by filtration, washed with EtOH (2×2 mL), Et₂O (2×2 mL) and dried under vacuum for several hours, yielding 0.285 g of a brown solid (96% yield). Elemental analysis for C₄₂H₃₀Cl₂Cu₂N₄O₄, MW=852.72 g/mol calculated: C, 59.16%; H, 3.55%; N, 6.57%. Found: C, 59.23%; H, 3.46%, N, 6.80%. IR spectrum, cm⁻¹ (ATR): 3058-2903, 1605, 1530, 1464-1430, 1319, 1270, 1194, 1149, 1035-978, 744.

### 3.6 Synthesis of [Cu₂(μ-sal(2)thn)₂]

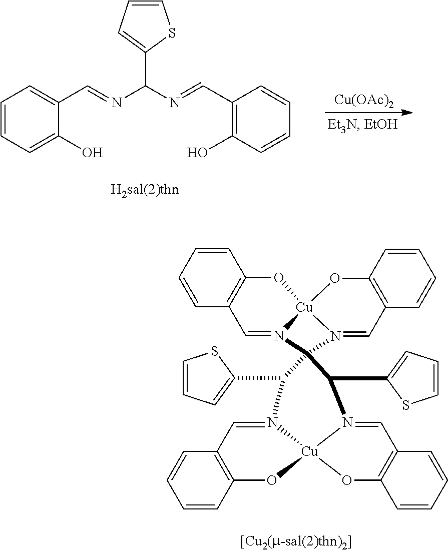

Et₃N (2.11 g, 20.8 mmol) was added to a yellow suspension of H₂sal(2)thn (1.699 g, 5.051 mmol) in 75 mL of EtOH. Copper acetate monohydrate (1.012 g, 5.070 mmol) was then added obtaining a colour change to brown in a few minutes. After 3 h under stirring at RT, the obtained dark brown solid was filtered, washed with EtOH (2×10 mL) and iPr₂O (2×10 mL) and dried under vacuum for several hours, yielding 1.398 g of the title compound (70%). Elemental analysis for C₃₈H₂₈Cu₂N₄O₄S₂, MW=795.88, calculated: C, 57.35%, H, 3.55%, N, 7.04%, S 8.06%. Found: C, 57.25%, H, 3.54%, N, 7.03%, S 8.19%. IR spectrum, cm⁻¹ (ATR): 3084, 3016, 2894, 1602, 1531, 1465, 1437, 1393, 1344, 1320, 1241, 1190, 1145, 1123, 1033, 973, 750, 735, 707. Mass spectrum (ESI⁺) in MeCN, m/z (intensity %): calculated for [M+H]+: 795.0223; found: 795.0217 (100).

### 3.7 Synthesis of [Cu₂(μ-sal(3)thn)₂]

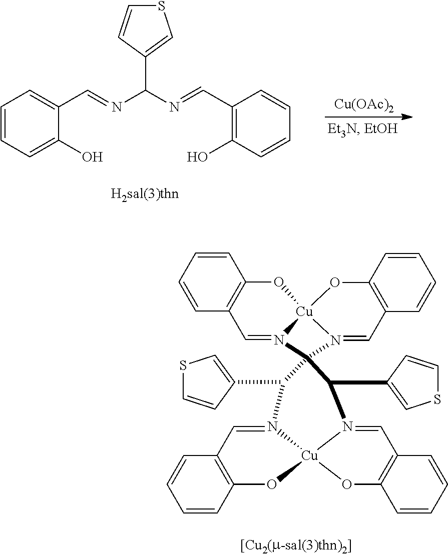

Et₃N (1.81 g, 17.9 mmol) was added to a yellow suspension of H₂sal(2)thn (1.706 g, 5.073 mmol) in 75 mL of EtOH. Copper acetate monohydrate (1.012 g, 5.072 mmol) was then added obtaining a colour change to brown in a few minutes. After 3 h under stirring at RT, the obtained dark brown solid was filtered, washed with EtOH (2×10 mL) and iPr₂O (2×10 mL) and dried under vacuum for several hours, yielding 1.695 g of the title compound (84%). Elemental analysis for C₃₈H₂₈Cu₂N₄O₄S₂, MW=795.88, calculated: C, 57.35%, H, 3.55%, N, 7.04%, S 8.06%. Found: C, 57.11%, H, 3.52%, N, 7.10%, S 8.06%. IR spectrum, cm¹ (ATR): 3088, 3017, 2898, 1604, 1532, 1465, 1439, 1393, 1343, 1320, 1192, 1145, 1123, 1033, 974, 845, 751, 732. Mass spectrum (ESI⁺) in MeCN, m/z (intensity %): calculated for [M+H]⁺: 795.0223; found: 795.0200 (100).

### 3.8.1 Synthesis of [Cu₂(μ-(R)-salC1myr)₂]

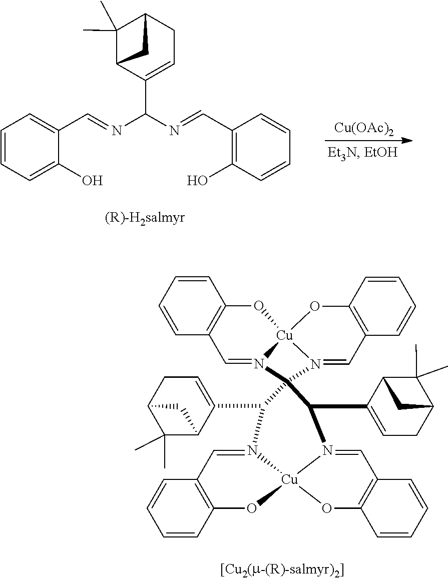

Solid Cu(OAc)₂·H₂O (0.09931 g, 0.497 mmol) was added to a yellow suspension of (R)—H₂salC1myr (0.20174 g, 0.539 mmol) in EtOH (8 mL) and NEt₃ (0.11112 g, 1.10 mmol) and the resulting dark green/brown mixture was stirred at RT for 3 h. The formed dark brown solid was filtered, washed with EtOH (3×3 mL) and iPr₂O (3×3 mL) and dried under vacuum for 2 h. Yield: 0.10927 g (50%). Further title compound was isolated upon storing the reaction mixture in the fridge for 4 d (0.06016 g, 28%). Elemental analysis calcd (%) for C₄₈H₄₈Cu₂N₄O₄ (872.03): C, 66.11, H, 5.55, N, 6.43. Found: C, 66.22, H, 5.90, N, 6.30. IR-ATR (cm⁻¹): 2970, 2918, 2864, 1600, 1532, 1464, 1445, 1325, 1194, 1145, 1051, 979, 917, 849, 752. Mass spectrum (ESI⁺) in MeCN, m/z (intensity %): calculated for [M+H]+: 871.2346; found: 871.2319 (100).

### 3.8.2 Synthesis of [Cu₂(μ-(R)-salC1myr)₂]

Solid Cu(OAc)₂·H₂O (0.0544 g, 0.273 mmol) was added to a yellow suspension of (R)—H₂salC1myr (0.1023 g, 0.273 mmol) in EtOH (4 mL) and NEt₃ (0.0566 g, 0.559 mmol) and the resulting dark green/brown mixture was stirred at RT for 3 h. The formed dark brown solid was filtered, washed with EtOH (3×3 mL) and iPr₂O (3×3 mL) and dried under vacuum for 2 h. Yield: 0.09696 g (82%). Elemental analysis calcd (%) for C₄₈H₄₈Cu₂N₄O₄·55H₂O (881.94): C, 65.37, H, 5.61, N, 6.35. Found: C, 65.32, H, 5.56, N, 6.35. IR-ATR (cm⁻¹): 3015, 2974, 2926, 2827, 1608, 1533, 1464, 1440, 1348, 1318, 1246, 1194, 1147, 971, 925, 750.

### 3.9 Synthesis of [Cu₂(μ-(4-OMe)salben)₂]

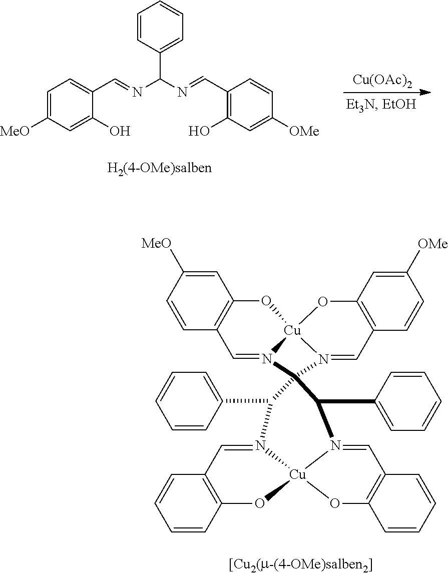

Et₃N (0.109 g, 1.08 mmol) was added to a yellow suspension of H₂(4-OMe)salben (0.2333 g, 0.5974 mmol) in 8 mL of EtOH. Solid Cu(OAc)₂H₂O (0.1216 g, 0.6090 mmol) was then added obtaining a colour change to brown in a few minutes. The reaction mixture was left under magnetic stirring for 3 h at RT, and then the precipitate was isolated by filtration, washed with EtOH (2×2 mL) and dried under vacuum for several hours. Yield: 0.1198 g of a light brown solid (44%). Elemental analysis calcd (%) for C₄₆H₄₀Cu₂N₄O₈ (903.94): C, 61.12, H, 4.46, N, 6.20. Found: C 61.15, H, 4.57, N, 5.86. IR-ATR (cm⁻¹): 3002, 2936, 2839, 1592, 1520, 1485, 1439, 1314, 1217, 1117, 1025, 975, 832.

### 3.10 Synthesis of [Cu₂(μ-(5-OMe)salben)₂]

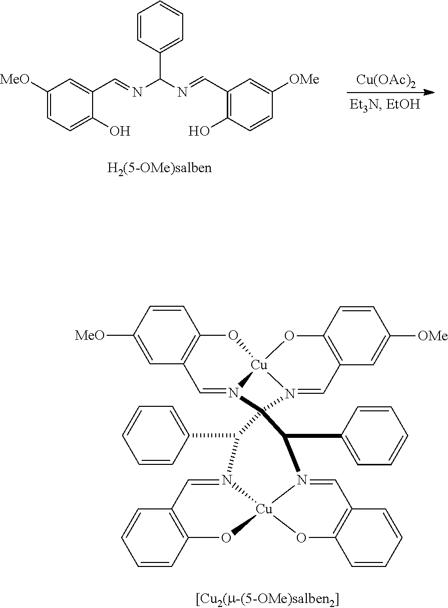

Et₃N (54.5 mg, 0.538 mmol) was added to a yellow suspension of H₂(5-OMe)salben (0.1005 g, 0.2575 mmol) in 4 mL of EtOH. Solid Cu(OAc)₂H₂O (0.05171 g, 0.2590 mmol) was then added obtaining a colour change to brown in a few minutes. The reaction mixture was left under magnetic stirring for 3 h at RT, and then the precipitate was isolated by filtration, washed with EtOH (2×2 mL) and dried under vacuum for several hours. Yield: 0.08951 g of a brown solid (77%). Elemental analysis calcd (%) for C₄₆H₄₀Cu₂N₄O₈ (903.94): C, 61.12, H, 4.46, N, 6.20. Found: C, 60.97, H, 4.43, N, 6.13. IR-ATR (cm⁻¹): 3062, 2928, 2827, 1623, 1592, 1532, 1456, 1307, 1215, 1157, 1026, 975, 813, 700. Mass spectrum (ESI⁺) in MeCN, m/z (intensity %): calculated for [M+H]+: 903.1517; found: 903.1331 (100).

### 3.11 Synthesis of [Cu₂(μ-(5-F)salben)₂]

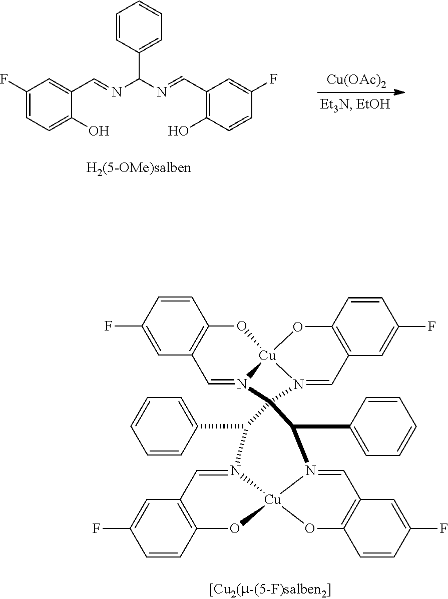

Et₃N (57.4 mg, 0.57 mmol) was added to a yellow suspension of H₂(5-F)salben (0.1004 g, 0.2742 mmol) in 4 mL of EtOH. Solid Cu(OAc)₂·H₂O (0.0564 g, 0.282 mmol) was then added obtaining a colour change to brown in a few minutes. The reaction mixture was left under magnetic stirring for 3 h at RT, and then the precipitate was isolated by filtration, washed with EtOH (2×2 mL) and dried under vacuum for several hours. Yield: 0.0945 g of a brown solid (81%). Elemental analysis calcd (%) for C₄₂H₂₈Cu₂F₄N₄O₄ (855.80): C, 58.95, H, 3.30, N, 6.53. Found: C, 58.91, H, 3.17, N, 6.53. IR-ATR (cm¹): 3053, 3004, 2897, 1607, 1538, 1457, 1393, 1310, 1241, 1209, 1141, 1002, 925, 865, 812.

### 3.12 Synthesis of [Cu₂(μ-(5-Cl)salben)₂]

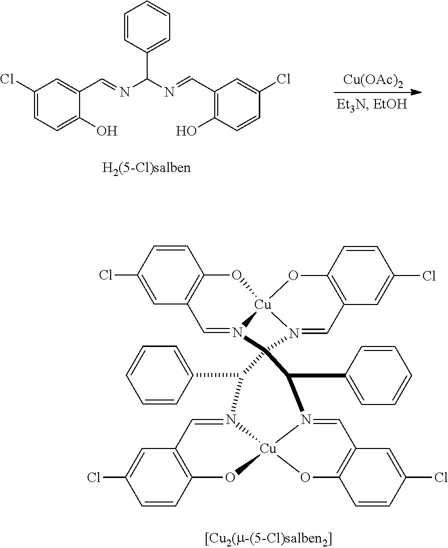

Et₃N (105.3 mg, 1.04 mmol) was added to a yellow suspension of H₂(5-Cl)salben (0.2015 g, 0.5048 mmol) in 8 mL of EtOH. Solid Cu(OAc)₂H₂O (0.10236 g, 0.5127 mmol) was then added obtaining a colour change to brown in a few minutes. The reaction mixture was left under magnetic stirring for 3 h at RT, and then the precipitate was isolated by filtration, washed with EtOH (2×2 mL) and dried under vacuum for several hours. Yield: 0.2074 g of a brown solid (89%). Elemental analysis calcd (%) for C₄₂H₂₈Cu₂Cl₄N₄O₄ (921.60): C, 54.34, H, 3.06, N, 6.08. Found: C, 54.37, H, 3.03, N, 5.99. IR-ATR (cm⁻¹): 3061, 2899, 1609, 1521, 1449, 1309, 1172, 1099, 976, 941, 876, 772. Mass spectrum (ESI⁺) in MeCN, m/z (intensity %): calculated for [M+H]⁺: 942.9325; found: 942.9382 (100).

### 3.13 Synthesis of [Cu₂(μ-(5-Me)salben)₂]

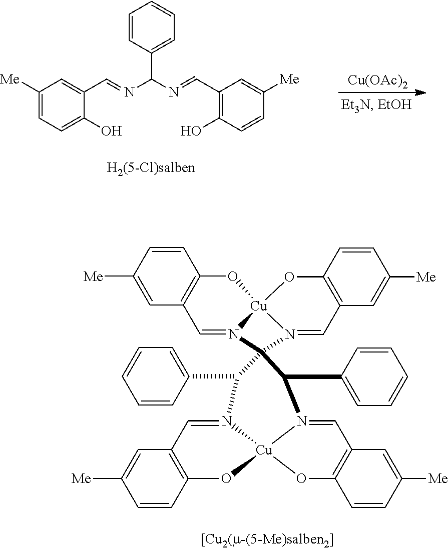

Et₃N (61.7 mg, 0.609 mmol) was added to a yellow suspension of H₂(5-Me)salben (0.1019 g, 0.2843 mmol) in 4 mL of EtOH. Solid Cu(OAc)₂H₂O (0.0568 g, 0.284 mmol) was then added obtaining a colour change to brown in a few minutes. The reaction mixture was left under magnetic stirring for 3 h at RT, and then the precipitate was isolated by filtration, washed with EtOH (2×2 mL) and dried under vacuum for several hours. Yield: 0.1001 g of a brown solid (84%). Elemental analysis calcd (%) for C₄₆H₄₀Cu₂N₄O₄ (839.94): C, 65.78, H, 4.80, N, 6.67. Found: C, 65.78, H, 4.75, N, 6.64. IR-ATR (cm⁻¹): 3059, 3004, 2911, 2853, 1619, 1529, 1468, 1382, 1313, 1251, 1137, 1045, 974, 824, 759. Mass spectrum (ESI⁺) in MeCN, m/z (intensity %): calculated for [M+H]+: 839.1720; found: 839.1714 (100).

### 3.14 Synthesis of [Cu₂(μ-(4-OMe)sal(2-Cl)ben)₂]

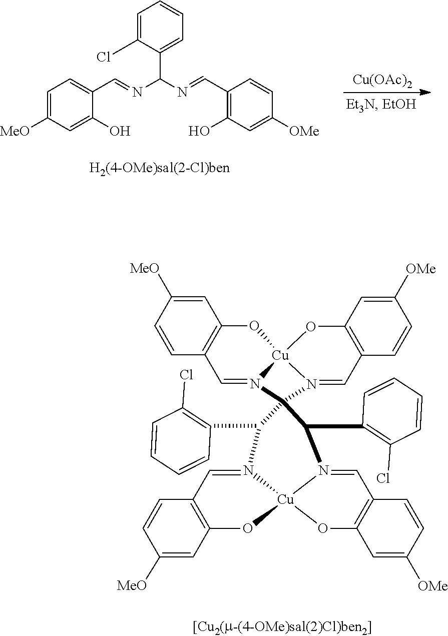

Et₃N (54.5 mg, 0.538 mmol) was added to a yellow suspension of H₂(4-OMe)sal(2-Cl)ben (0.1099 g, 0.2587 mmol) in 4 mL of EtOH. Solid Cu(OAc)₂·H₂O (0.05256 g, 0.2633 mmol) was then added obtaining a colour change to brown in a few minutes. The reaction mixture was left under magnetic stirring for 3 h at RT, and then the precipitate was isolated by filtration, washed with EtOH (2×2 mL) and dried under vacuum for several hours. Yield: 0.0672 g of a brown solid (52%). Elemental analysis calcd (%) for C₄₆H₃₈C₁₂Cu₂N₄O₈·H₂O (990.84): C, 55.76, H, 4.07, N, 5.65. Found: C, 55.85, H, 3.85, N, 5.45. IR-ATR (cm⁻¹): 3059, 3004, 2911, 2853, 1619, 1595, 1529, 1468, 1382, 1313, 1251, 1215, 1163, 1137, 1045, 974, 824, 759.

### 3.15 Synthesis of [Cu₂(μ-(5-OMe)sal(2-Cl)ben)₂]

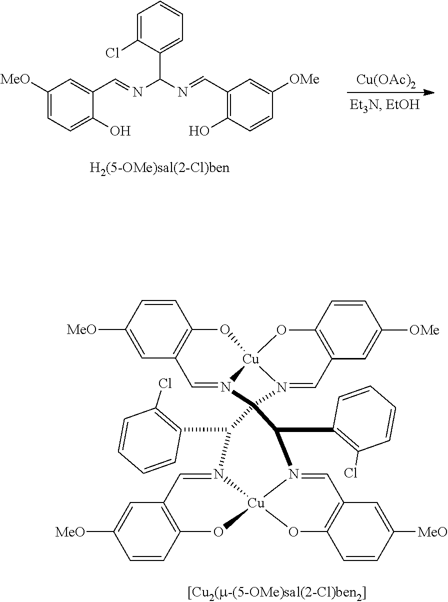

Et₃N (36.4 mg, 0.359 mmol) was added to a yellow suspension of H₂(5-OMe)sal(2-Cl)ben (0.7146 g, 0.1685 mmol) in 3 mL of EtOH. Solid Cu(OAc)₂·H₂O (0.0330 g, 0.165 mmol) was then added obtaining a colour change to brown in a few minutes. The reaction mixture was left under magnetic stirring for 3 h at RT, and then the precipitate was isolated by filtration, washed with EtOH (2×2 mL) and dried under vacuum for several hours. Yield: 0.07628 g of a brown solid (95%). Elemental analysis calcd (%) for C₄₆H₃₈C₁₂Cu₂N₄O₈ (972.82): C, 56.79, H, 3.94, N, 5.76. Found: C, 56.85, H, 4.00, N, 5.73. IR-ATR (cm⁻¹): 2831, 1624, 1594, 1532, 1470, 1307, 1218, 1159, 1031, 973, 952, 824, 755.

### 3.16 Synthesis of [Cu₂(μ-(5-OMe)sal(2-OMe)ben)₂]

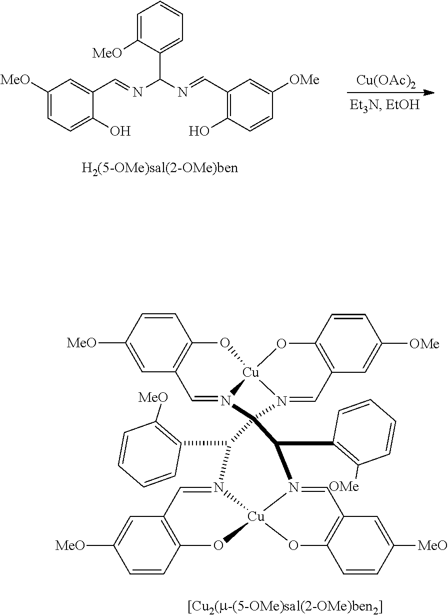

Et₃N (98.0 mg, 0.969 mmol) was added to a yellow suspension of H₂(5-OMe)sal(2-OMe)ben (0.2004 g, 0.4763 mmol) in 8 mL of EtOH. Solid Cu(OAc)₂·H₂O (0.0951 g, 0.0968 mmol) was then added obtaining a colour change to brown in a few minutes. The reaction mixture was left under magnetic stirring for 3 h at RT, and then the precipitate was isolated by filtration, washed with EtOH (2×2 mL) and dried under vacuum for several hours. Yield: 0.2091 g of a brown solid (91%). Elemental analysis calcd (%) for C₄₈H₄₄Cu₂N₄O₁₀ (963.99): C, 59.81, H, 4.60, N, 5.81. Found: C, 60.28, H, 4.51, N, 5.82. IR-ATR (cm⁻¹): 3053, 2832, 1622, 1597, 1533, 1459, 1392, 1306, 1246, 1219, 1158, 1207, 953, 824, 754. Mass spectrum (ESI⁺) in MeCN, m/z (intensity %): calculated for [M+H]⁺: 963.1728; found: 963.1677 (100).

### 3.17 Synthesis of [Cu₂(μ-(5-Cl)sal(2-OMe)ben)₂]

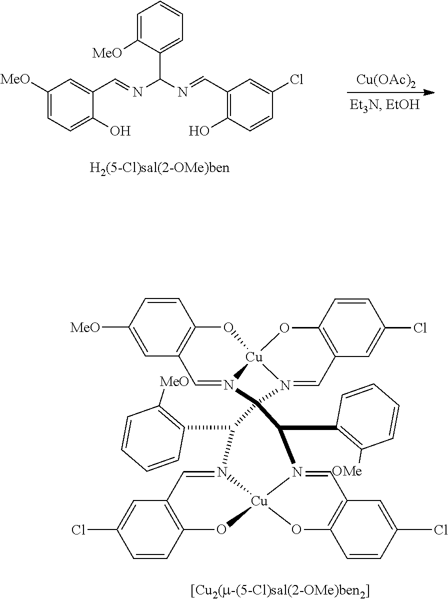

Et₃N (98.0 mg, 0.969 mmol) was added to a yellow suspension of H₂(5-Cl)sal(2-OMe)ben (0.1929 g, 0.1685 mmol) in 8 mL of EtOH. Solid Cu(OAc)₂·H₂O (0.0940 g, 0.471 mmol) was then added obtaining a colour change to brown in a few minutes. The reaction mixture was left under magnetic stirring for 3 h at RT, and then the precipitate was isolated by filtration, washed with EtOH (2×2 mL) and dried under vacuum for several hours. Yield: 0.2088 g of a brown solid (94%). Elemental analysis calcd (%) for C₄₄H₃₂Cl₄Cu₂N₄O₆ (981.65): C, 53.84, H, 3.29, N, 5.71. Found: C, 53.74, H, 3.26, N, 5.60. IR-ATR (cm⁻¹): 2960, 2839, 1611, 1520, 1483, 1457, 1311, 1241, 1172, 1043, 982, 823, 740.

### 3.18 Synthesis of [Cu₂(μ-(5-Cl)sal(2-NO₂)ben)₂]

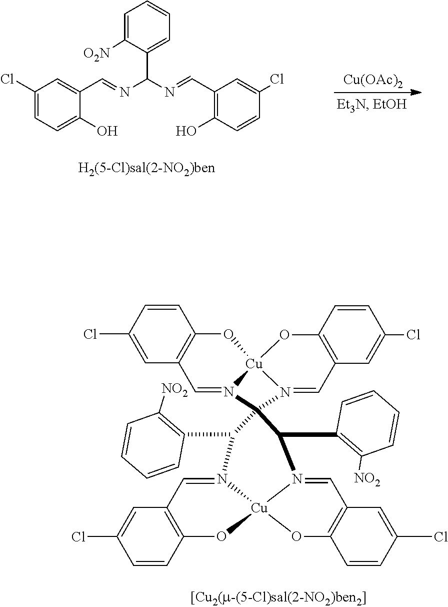

Et₃N (94.4 mg, 0.933 mmol) was added to a yellow suspension of H₂(5-Cl)sal(2-NO₂)ben (0.2046 g, 0.460 mmol) in 8 mL of EtOH. Solid Cu(OAc)₂·H₂O (0.0898 g, 0.450 mmol) was then added obtaining a colour change to brown in a few minutes. The reaction mixture was left under magnetic stirring for 3 h at RT, and then the precipitate was isolated by filtration, washed with EtOH (2×2 mL) and dried under vacuum for several hours. Yield: 0.2123 g of a brown solid (93%). Elemental analysis calcd (%) for C₄₂H₂₆C₁₄Cu₂N₆O₈ (1011.60): C, 49.87, H, 2.59, N, 8.31. Found: C, 49.63, H, 2.55, N, 8.00. IR-ATR (cm⁻¹): 1610, 1520, 1384, 1311, 1175, 1036, 978, 829, 765.

### 3.19 Synthesis of [Cu₂(μ-(5-OMe)sal(2-NO₂)ben)₂]

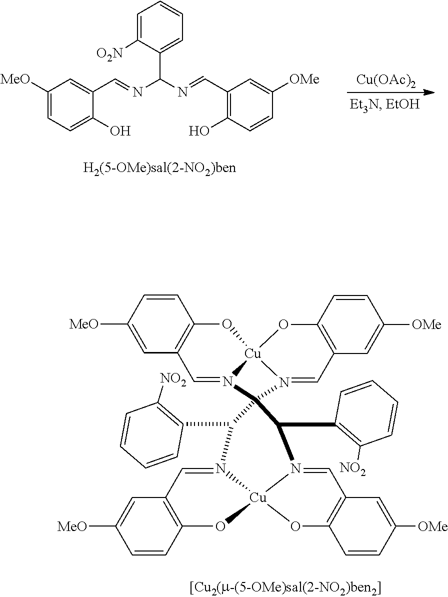

Et₃N (69.0 mg, 0.682 mmol) was added to a yellow suspension of H₂(5-OMe)sal(2-NO₂)ben (0.1435 g, 0.330 mmol) in 5.5 mL of EtOH. Solid Cu(OAc)₂·H₂O (0.0665 g, 0.333 mmol) was then added obtaining a colour change to brown in a few minutes. The reaction mixture was left under magnetic stirring for 3 h at RT, and then the precipitate was isolated by filtration, washed with EtOH (2×2 mL) and dried under vacuum for several hours. Yield: 0.141 g of a brown solid (82%). Elemental analysis calcd (%) for C₄₆H₃₈Cu₂N₆O₁₂·EtOH (1040.00): C, 55.44, H, 4.29, N, 8.08. Found: C, 55.06, H, 3.94, N, 7.81. IR-ATR (cm⁻¹): 2853, 1625, 1596, 1525, 1467, 1339, 1302, 1271, 1218, 1160, 1032, 954, 811, 766.

### 3.20 Synthesis of [Cu₂(μ-(sal(2-NO₂)ben)₂]

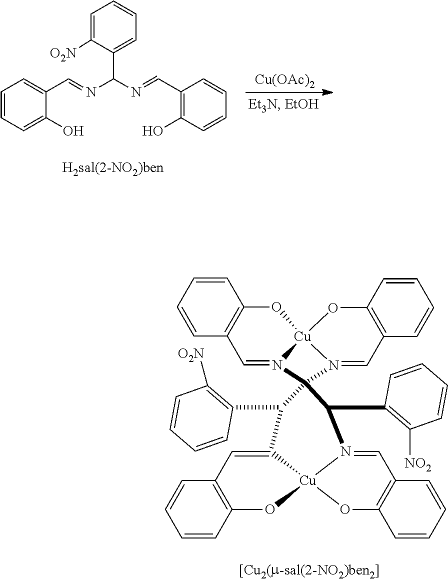

Et₃N (58.1 mg, 0.574 mmol) was added to a yellow suspension of H₂sal(2-NO₂)ben (0.100 g, 0.266 mmol) in 4 mL of EtOH. Solid Cu(OAc)₂H₂O (0.050 g, 0.275 mmol) was then added obtaining a colour change to brown in a few minutes. The reaction mixture was left under magnetic stirring for 3 h at RT, and then the precipitate was isolated by filtration, washed with EtOH (2×2 mL) and dried under vacuum for 1 h. Yield: 0.1022 g of a brown solid (43.9%). Elemental analysis calcd (%) for C₄₂H₃₀Cu₂N₆O₈ (873.82): C, 57.73, H, 3.46, N, 9.62, O 14.65. Found: C, 57.5, H, 3.6, N, 9.6, O 14.7. IR-ATR (cm⁻¹): 3039, 3021, 2940, 2904, 1605, 1522, 1464, 1443, 1339, 1319, 1194, 1147, 1126, 1027, 979, 851, 751, 741, 713. Mass spectrum (ESI⁺) in MeCN, m/z (intensity %): calculated for [M+CH₃CN+NH₄⁺]⁺: 931.1327; found: 931.1322 (100).

### 4. C—N Coupling Reactions

### 4.1 Coupling of Iodobenzene with Pyrazole with Cs₂CO₃ as base

Under inert atmosphere, iodobenzene (215 μL, 1.93 mmol, 1.5 equiv) and pyrrole (90 μL, 1.29 mmol, 1 equiv) were added to a suspension of Cs₂CO₃ (839.2 mg, 2.58 mmol, 2 equiv) and [Cu₂(μ-salben)₂](5 mg, 6.45×10⁻³ mmol, 0.005 equiv) in 1 mL of MeCN in a 10 mL tube with screw cap. The tube was closed and heated with an oil bath at 82 °C and the reaction mixture was stirred for 16 h. The suspension was allowed to cool to RT and then the solvent was removed under reduced pressure. The obtained residue was treated with 10 mL of dichloromethane (DCM), the resulting suspension was filtered on a paper filter, and the remaining solid was washed with DCM (2×5 mL). The organic phase was concentrated under reduced pressure to obtain the crude product as a brown liquid that was purified via flash column chromatography on silica gel to afford 153 mg of the desired product (83% yield). The identity of the product was confirmed by comparison of the ¹H NMR spectrum with the commercially available sample. ¹H NMR, 298 K, CDCl₃, 400 MHz: δ (ppm) 7.45-7.38 (4H, m), 7.27-7.23 (1H, m), 7.09 (2H, t), 6.35 (2H, t).

### 4.2 Coupling of iodobenzene with pyrazole with K₃PO₄ as base

Under inert atmosphere, iodobenzene (720 μL, 6.47 mmol, 1.5 equiv) and pyrrole (300 μL, 4.29 mmol. 1 equiv) were added to a suspension of K₃PO₄ (17.2 mmol, 3.65 g, 4 equiv) and [Cu₂(μ-salben)₂](0.0429 mmol, 33.6 mg, 0.010 equiv) in 3 mL of MeCN in a 50 mL tube with screw cap. The tube was closed and heated with an oil bath at 82 °C and the reaction mixture was stirred for 16 h. The suspension was allowed to cool to RT and then the solvent was removed under reduced pressure. The obtained residue was treated with 10 mL of DCM, the obtained suspension was filtered on a paper filter, and the remaining solid was washed with DCM (2×5 mL). The organic phase was concentrated under reduced pressure to obtain the crude product as a brown liquid. The residual oil was purified via flash column chromatography on silica gel to afford 521 mg of the desired product (85% yield). The identity of the product was confirmed by comparison of the ¹H NMR spectrum with a commercially available sample. ¹H NMR, 298 K, CDCl₃, 400 MHz: δ (ppm) 7.45-7.38 (4H, m), 7.27-7.23 (1H, m), 7.09 (2H, t), 6.35 (2H, t).

### 4.3 General Procedure for the Screening of Solvents and Bases

Under inert atmosphere, a 10 mL tube was charged with the base and the catalyst. The solvent was added (1 mL), and iodobenzene and pyrrole were added to the resulting suspension. The tube was closed and heated at the indicated temperature. After the indicated time, the tube was allowed to cool to RT. The suspension was filtered with a syringe on a 0.45 μm PTFE filter. The solid on the filter was washed with the reaction solvent (3×3 mL). The filtrated was concentrated under reduced pressure to obtain the crude product. The external standard solution (1 mL of a 0.429 mol L⁻¹ CDCl₃ solution of dimethylsulfone) was added, 150 μL of the so-obtained solution were diluted to 700 μL with CDCl₃ in an NMR tube, and the ¹H NMR spectrum was registered to calculate the yield.

### 4.4 General Procedure for the Coupling of Aryl Halides with N-Nucleophiles

Under inert atmosphere, a screw-cap glass tube equipped with a stirring bar was charged with the base, the catalyst and the solvent. To the suspension, the N-nucleophile and the aryl halide were added. The tube was closed and heated at the indicated temperature for the indicated time. The reaction mixture was filtered over 0.45 μm PTFE Syringe-Filter and the filter washed with MeCN (1-2 mL). LC-MS sample was prepared from resulting solution. Optionally, the solvent was evaporated, and the crude product purified by chromatography for additional characterization.

### 4.5 Characterization Data for Product

| | | | Characterization |
|---|---|---|---|
| Aryl halide | N-Nucleophile | Product | data |
| 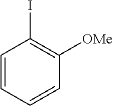 | 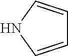 | 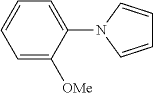 [SMILES: COc1ccccc1-n1cccc1] | MS (EI⁺): m/z 174.1 [M + 1]⁺ |
| 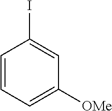 | 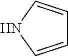 | 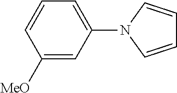 [SMILES: COc1cccc(-n2cccc2)c1] | MS (EI⁺): m/z 174.1 [M + 1]⁺ |
| 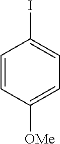 | 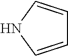 | 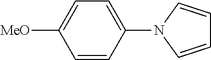 [SMILES: COc1ccc(-n2cccc2)cc1] | MS (EI⁺): m/z 174.1 [M + 1]⁺ |
| 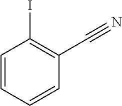 | 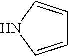 | 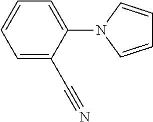 [SMILES: N#Cc1ccccc1-n1cccc1] | MS (EI⁺): m/z 169.1 [M + 1]⁺ |
| 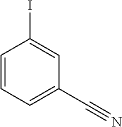 | 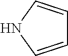 | 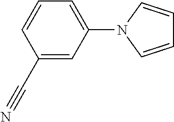 [SMILES: N#Cc1cccc(-n2cccc2)c1] | MS (EI⁺): m/z 169.1 [M + 1]⁺ |
| 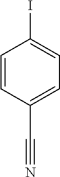 | 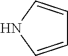 | 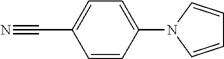 [SMILES: N#Cc1ccc(-n2cccc2)cc1] | MS (EI⁺): m/z 169.1 [M + 1]⁺ |
| 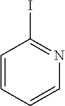 | 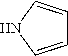 | 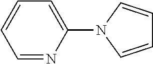 [SMILES: c1ccc(-n2cccc2)nc1] | MS (EI⁺): m/z 145.1 [M + 1]⁺ |
| 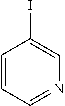 | 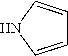 | 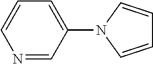 [SMILES: c1cncc(-n2cccc2)c1] | MS (EI⁺): m/z 145.1 [M + 1]⁺ |
| 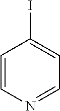 | 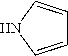 | 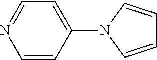 [SMILES: c1ccn(-c2ccncc2)c1] | MS (EI⁺): m/z 145.0 [M + 1]⁺ |
| 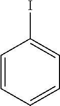 | 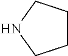 | 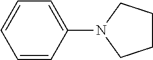 [SMILES: c1ccc(N2CCCC2)cc1] | MS (EI⁺): m/z 148.1 [M + 1]⁺ |
| 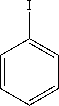 | 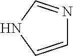 | 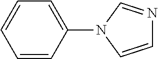 [SMILES: c1ccc(-n2ccnc2)cc1] | ¹H NMR, 298K, CDCl₃, 400 MHz: δ (ppm) 7.87 (s, 1 H), 7.51-7.47 (m, 2 H), 7.41-7.36 (m, 3 H), 7.30 (s, 1H), 7.22 (s, 1 H). |
| | | | MS (EI⁺): m/z 145.0 |
| | | | [M + 1]⁺ |
| 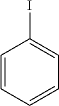 | 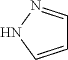 | 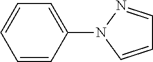 [SMILES: c1ccc(-n2cccn2)cc1] | MS (EI⁺): m/z 145.0 [M + 1]⁺ |
| 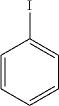 | 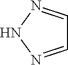 | 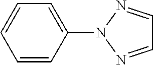 [SMILES: c1ccc(-n2nccn2)cc1] | MS (EI⁺): m/z 146.1 [M + 1]⁺ |
| 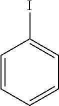 | 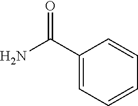 | 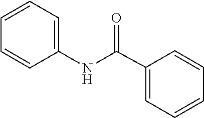 [SMILES: O=C(Nc1ccccc1)c1ccccc1] | MS (EI⁺): m/z 198.2 [M + 1]⁺ |
| 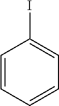 | 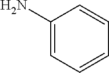 | 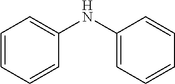 [SMILES: c1ccc(Nc2ccccc2)cc1] | MS (EI⁺): m/z 170.1 [M + 1]⁺ |
| 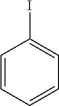 | 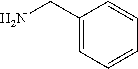 | 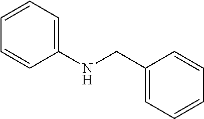 [SMILES: c1ccc(CNc2ccccc2)cc1] | MS (EI⁺): m/z 184.1 [M + 1]⁺ |
| 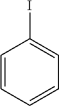 | 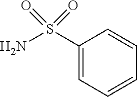 | 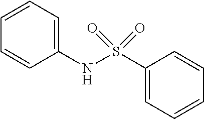 [SMILES: O=S(=O)(Nc1ccccc1)c1ccccc1] | MS (EI⁺): m/z 234.1 [M + 1]⁺ |
| 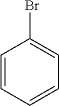 | 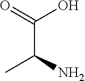 | 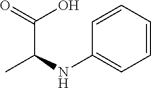 | MS (EI⁺): m/z 164.2 [M − 1]⁺ |
| 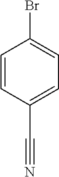 | 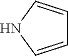 | 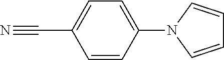 [SMILES: N#Cc1ccc(-n2cccc2)cc1] | ¹H NMR, 298K, CDCl₃, 600 MHz: δ (ppm) 7.73-7.75 (m, 1 H), 7.70-7.77 (m, 1 H), 7.48-7.54 (m, 2 H), 7.15-7.17 (m, 1 H), 7.15-7.19 (m, 1 H), 6.41-6.45 (m, 2 H) MS (EI⁺): m/z 168.1 [M]⁺ |
| 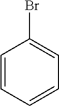 | 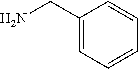 | 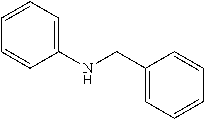 [SMILES: c1ccc(CNc2ccccc2)cc1] | ¹H NMR, 298K, CDCl₃, 600 MHz: δ (ppm) 7.43-7.48 (m, 1 H), 7.36-7.38 (m, 3 H), 7.29-7.33 (m, 1 H), 7.18-7.23 (m, 2 H), 6.75 (tt, J = 7.3, 1.1 Hz, 1 H), |
| | | | 6.67-6.69 (m, 2 H), |
| | | | 4.36 (s, 2 H), 3.91- |
| | | | 4.19 (m, 1 H) |
| | | | MS (EI⁺): m/z 183.1 |
| | | | [M]⁺ |
| 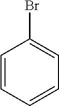 | 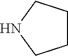 | 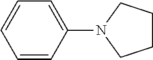 [SMILES: c1ccc(N2CCCC2)cc1] | MS (EI⁺): m/z 148.0 [M + 1]⁺ |
| 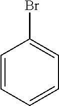 | 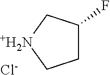 | 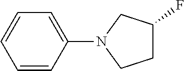 [SMILES: F[C@@H]1CCN(c2ccccc2)C1] | MS (EI⁺): m/z 166.0 [M + 1]⁺ |
| 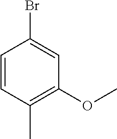 | 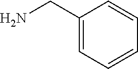 | 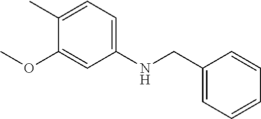 [SMILES: COc1cc(NCc2ccccc2)ccc1C] | MS (EI⁺): m/z 228.1 [M + 1]⁺ |
| 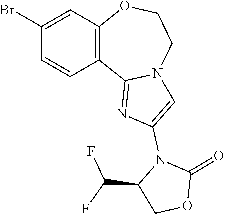 [SMILES: O=C1OC[C@@H](C(F)F)N1c1cn2c(n1)-c1ccc(Br)cc1OCC2] | 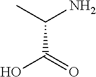 | 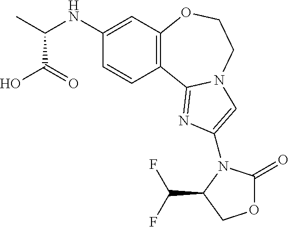 [SMILES: C[C@H](Nc1ccc2c(c1)OCCn1cc(N3C(=O)OC[C@H]3C(F)F)nc1-2)C(=O)O] | MS ESI+: m/z 409.1 [M + 1]⁺ |

1-15. (canceled)

16. A dinuclear copper(II) complex of formula I 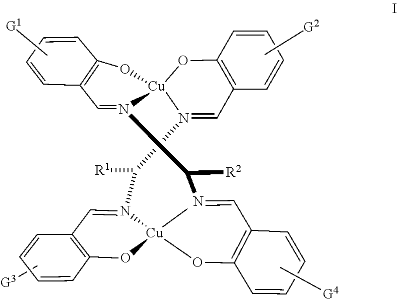
wherein
R1 and R2 are the same or different and are optionally substituted C1-12-alkyl, aryl or heteroaryl, or hydrogen and
G1, G2, G3 and G4 are the same or different and stand for hydrogen or one or more substituents selected from C1-12-alkyl, C1-12-alkoxy, halogen, halogen C1-12-alkyl, mono- or di-C1-12-alkyl amino, carboxyl, C1-12-alkoxy carbonyl or nitro or G1, G2, G3 and G4, independently of each other, together with the phenyl ring they are attached to form a fused aryl ring with two or three rings, which are optionally substituted with one or more substituents selected from C1-6-alkyl, C1-6-alkoxy, halogen, mono- or di-C1-6-alkyl amino or nitro;
R1 and R2 are the same and are substituents of the formula IIa, IIb or IIc 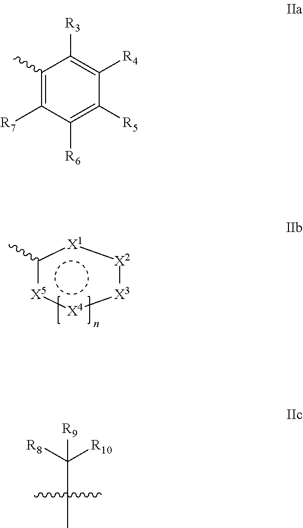
R3, R4, R5, R6 and R7 are the same or different and are hydrogen, C1-12-alkyl, C1-12-alkoxy, halogen, halogen-C1-12-alkyl, mono- or di-C1-12-alkyl amino, nitro or R3 and R4 or R4 and R5 or R5 and R6 or R6 and R7 together with the phenyl ring they are attached to form a fused aryl ring with two or three rings, which are optionally substituted with one or more substituents selected from C1-6-alkyl, C1-6-alkoxy, halogen, mono- or di-C1-6-alkyl amino or nitro.
X1, X2, X3, X4 and X5 are the same or different and represent at least one heteroatom selected from nitrogen, oxygen or sulphur;
n is 0 or 1;
R8, R9 and R10 are the same or different and are hydrogen, C1-12-alkyl, halogen C1-12-alkyl, hydroxy, hydroxy-C1-12-alkyl, halogen or R8 and R9 together form a saturated carbocycle or a heterocycle or R8, R9 and R10 together form an unsaturated carbocycle,
with the proviso that compounds with R1 and R2 being hydrogen, or R1 and R2 having the formula IIa and wherein R3, R4, R6 and R7 are hydrogen, while R5 is hydrogen, chlorine, methyl, or nitro, are excluded.

17. The dinuclear copper(II) complex of claim 16, wherein R1 and R2 are the same and are substituents of the formula IIa, IIb or IIc 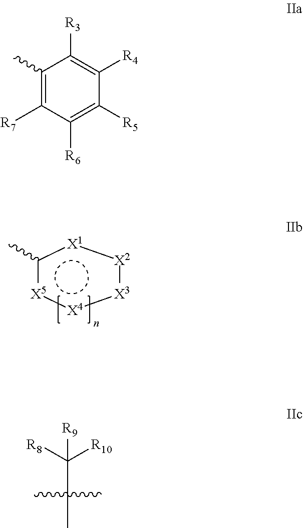
wherein
R3, R4, R5, R6 and R7 are the same or different and are hydrogen, C1-12-alkyl, C1-12-alkoxy, halogen, halogen-C1-12-alkyl, mono- or di-C1-12-alkyl amino, nitro or R3 and R4 or R4 and R5 or R5 and R6 or R6 and R7 together with the phenyl ring they are attached to form a fused aryl ring with two or three rings, which are optionally substituted with one or more substituents selected from C1-6-alkyl, C1-6-alkoxy, halogen, mono- or di-C1-6-alkyl amino or nitro,
X1, X2, X3, X4 and X5 are the same or different and represent at least one heteroatom selected from nitrogen, oxygen or sulphur;
n is 0 or 1;
R8, R9 and R10 are the same or different and are hydrogen, C1-12-alkyl, halogen C1-12-alkyl, hydroxy, hydroxy-C1-12-alkyl, halogen or R8 and R9 together form a saturated carbocycle or a heterocycle or R8, R9 and R10 together form an unsaturated carbocycle.

18. The dinuclear copper(II) complex of claim 16, wherein the aryl halogenide has the formula III 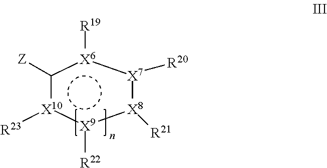
wherein
X6, X7, X8, X9 and X10 are the same or different and represent carbon or at least one heteroatom selected from nitrogen, oxygen or sulphur;
R19, R20, R21, R22 and R23 are the same or different and are hydrogen, C1-12-alkyl, C2-12-alkenyl, C2-12-alkinyl, C1-12-alkoxy, halogen, halogen-C1-12-alkyl, mono- or di-C1-12-alkyl amino or cyano, or R19 and R20 or R20 and R21 or R21 and R22 or R22 and R23 together with the ring they are attached to form a fused aryl ring with two or three rings, which are optionally substituted with one or more substituents selected from C1-6-alkyl, C1-6-alkoxy, halogen, mono- or di-C1-6-alkyl amino or nitro;
n is 0 or 1 and
Z is a halogen atom.

19. The dinuclear copper(II) complex of claim 16, wherein the aryl halogenide has the formula III 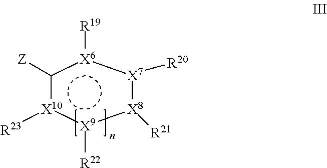
wherein
X6, X7, X8, X9 and X10 are the same or different and represent carbon or at least one heteroatom selected from nitrogen, oxygen or sulphur;
R19, R20, R21, R22 and R23 are the same or different and are hydrogen, C1-12-alkyl, C2-12-alkenyl, C2-12-alkinyl, C1-12-alkoxy, halogen, halogen-C1-12-alkyl, mono- or di-C1-12-alkyl amino or cyano, or R19 and R20 or R20 and R21 or R21 and R22 or R22 and R23 together with the ring they are attached to form a fused aryl ring with two or three rings, which are optionally substituted with one or more substituents selected from C1-6-alkyl, C1-6-alkoxy, halogen, mono- or di-C1-6-alkyl amino or nitro;
n is 0 or 1 and
Z is a halogen atom.

20. The dinuclear copper(II) complex of claim 16, wherein the dinuclear copper(II) complex is selected from the group consisting of

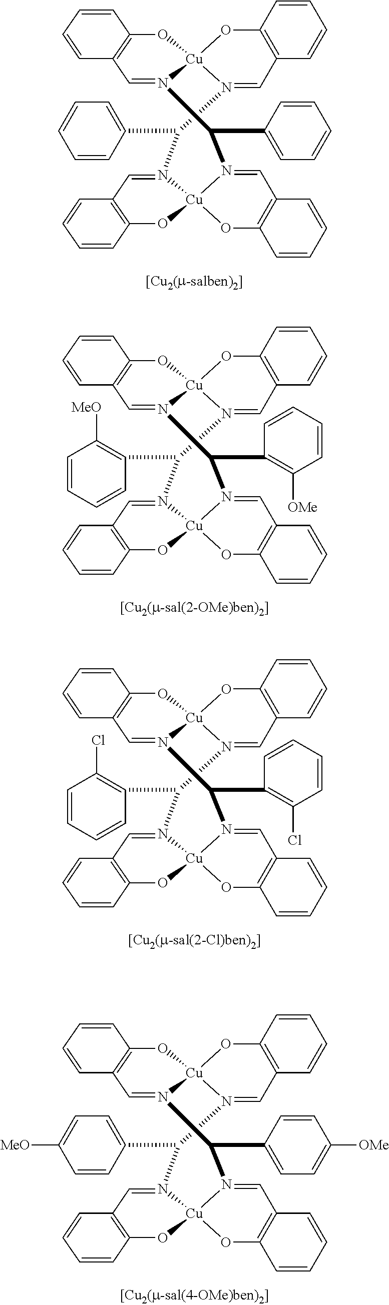

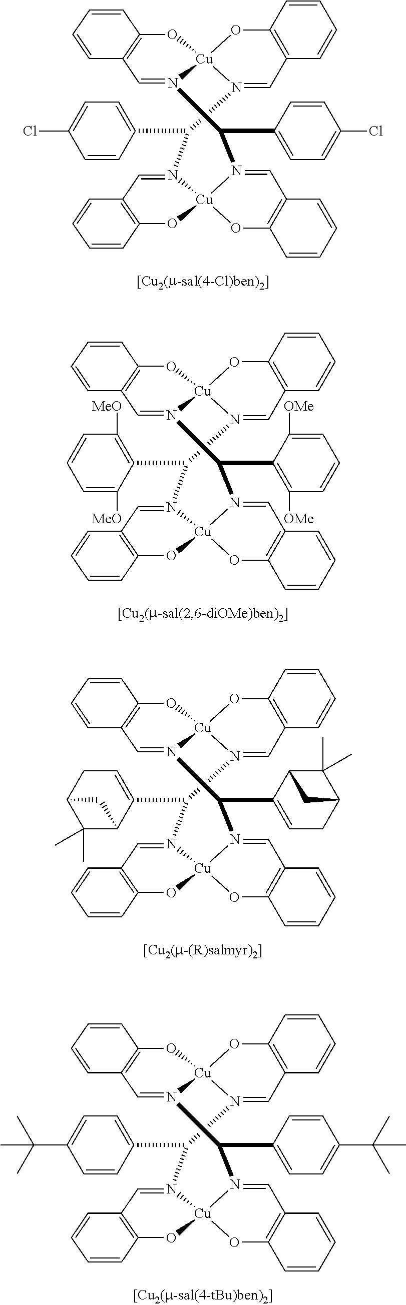

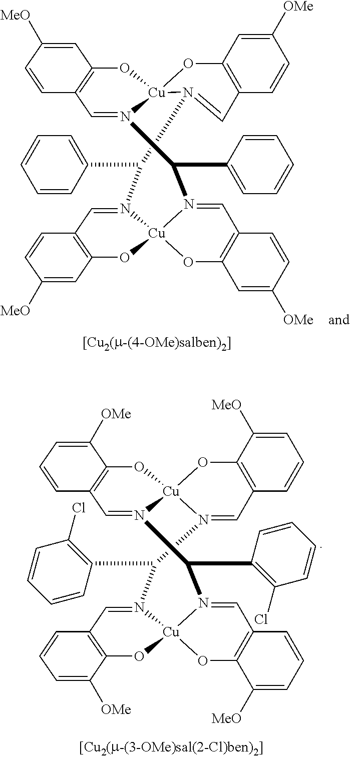

21. A process for the coupling of aryl halogenides with an N-nucleophile compound, the process comprising contacting the aryl halogenides and N-nucleophile compound with one or more dinuclear copper(II) complexes bearing substituted salben-type ligands of the formula I 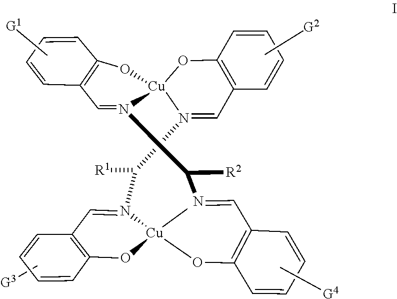
wherein R1 and R2 are the same or different and are optionally substituted C1-12-alkyl, aryl or heteroaryl, or hydrogen and
G1, G2, G3 and G4 are the same or different and hydrogen or one or more substituents selected from C1-12-alkyl, C1-12-alkoxy, halogen C1-12-alkyl, halogen, mono- or di-C1-12-alkyl amino, carboxyl, C1-12 alkoxy carbonyl or nitro or G1, G2, G3 and G4, independently of each other, together with the phenyl ring they are attached to form a fused aryl ring with two or three rings, which are optionally substituted with one or more substituents selected from C1-6-alkyl, C1-6-alkoxy, halogen, mono- or di-C1-6-alkyl amino or nitro.

22. The process of claim 22, wherein R1 and R2 are the same and are substituents of the formula IIa, IIb or IIc 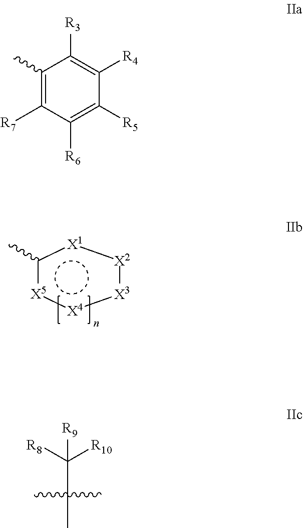
wherein
R3, R4, R5, R6 and R7 are the same or different and are hydrogen, C1-12-alkyl, C1-12-alkoxy, halogen, halogen-C1-12-alkyl, mono- or di-C1-12-alkyl amino, nitro or R3 and R4 or R4 and R5 or R5 and R6 or R6 and R7 together with the phenyl ring they are attached to form a fused aryl ring with two or three rings, which are optionally substituted with one or more substituents selected from C1-6-alkyl, C1-6-alkoxy, halogen, mono- or di-C1-6-alkyl amino or nitro,
X1, X2, X3, X4 and X5 are the same or different and represent at least one heteroatom selected from nitrogen, oxygen or sulphur;
n is 0 or 1;
R8, R9 and R10 are the same or different and are hydrogen, C1-12-alkyl, halogen C1-12-alkyl, hydroxy, hydroxy-C1-12-alkyl, halogen or R8 and R9 together form a saturated carbocycle or a heterocycle or R8, R9 and R10 together form an unsaturated carbocycle.

23. The process of claim 22, wherein the aryl halogenide has the formula III 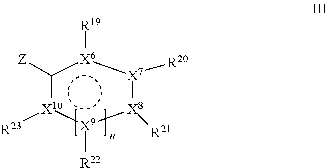
wherein
X6, X7, X8, X9 and X10 are the same or different and represent carbon or at least one heteroatom selected from nitrogen, oxygen or sulphur;
R19, R20, R21, R22 and R23 are the same or different and are hydrogen, C1-12-alkyl, C2-12-alkenyl, C2-12-alkinyl, C1-12-alkoxy, halogen, halogen-C1-12-alkyl, mono- or di-C1-12-alkyl amino or cyano, or R19 and R20 or R20 and R21 or R21 and R22 or R22 and R23 together with the ring they are attached to form a fused aryl ring with two or three rings, which are optionally substituted with one or more substituents selected from C1-6-alkyl, C1-6-alkoxy, halogen, mono- or di-C1-6-alkyl amino or nitro;
n is 0 or 1 and
Z is a halogen atom.

24. The process of claim 22, wherein the N-nucleophile is selected from 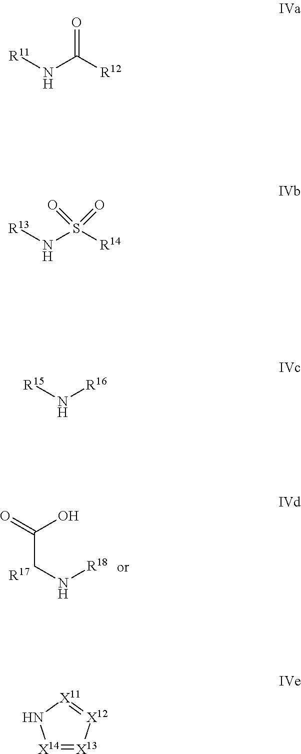
wherein
R11, R12, R13, R14, R15, R16, R17 and R18 are the same or different and are hydrogen, C1-12-alkyl, C1-12-alkoxy, aryl, halogen, halogen-C1-12-alkyl, nitro, hydroxy, mono- or di-C1-12-alkyl amino or R15 and R16 together with the nitrogen they are attached to, form a saturated heterocycle 5- or 6-member heterocycle;
X11, X12, X13 and X14 are the same or different and represent a carbon atom optionally substituted with C1-12-alkyl, aryl-C1-12-alkyl, C1-6-alkoxy, halogen, halogen-Cl-12-alkyl and at least one further heteroatom selected from nitrogen, oxygen or sulphur.

25. The process of claim 22, wherein the dinuclear copper(II) complex is selected from the group consisting of

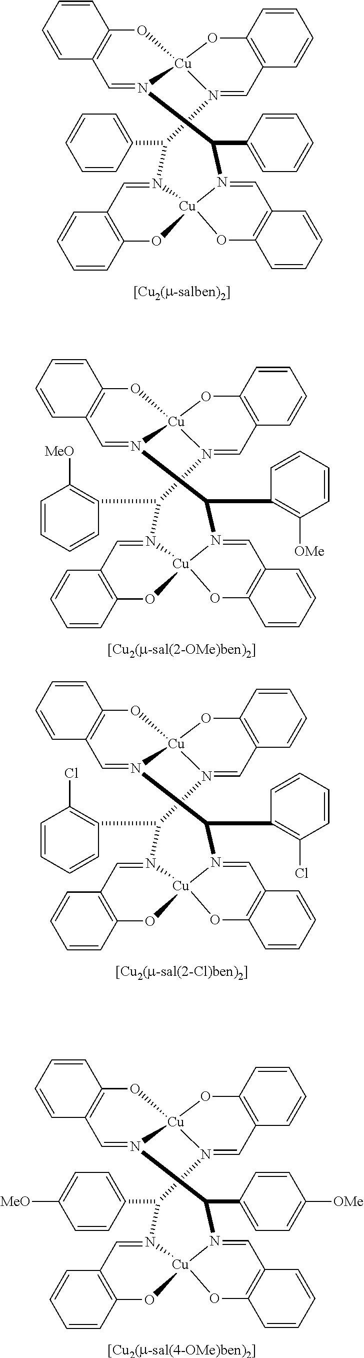

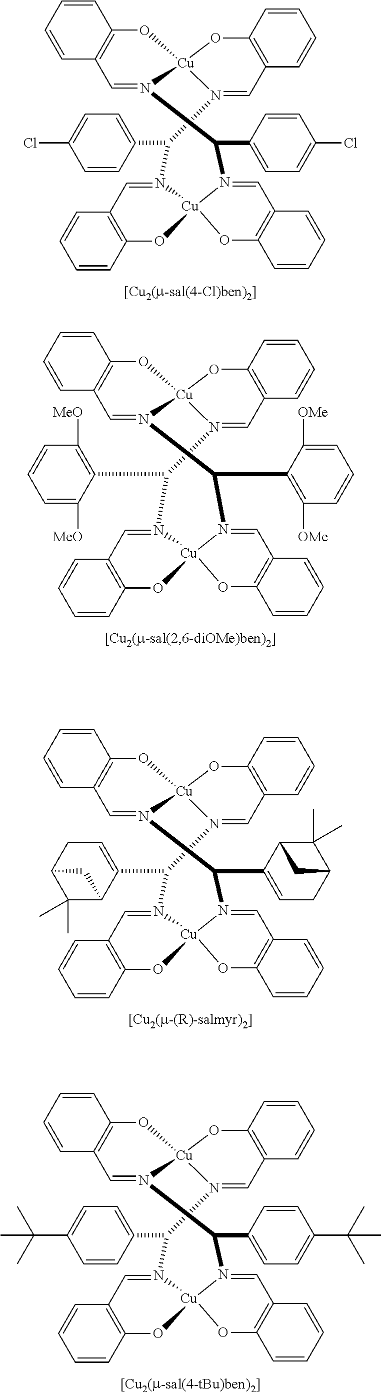

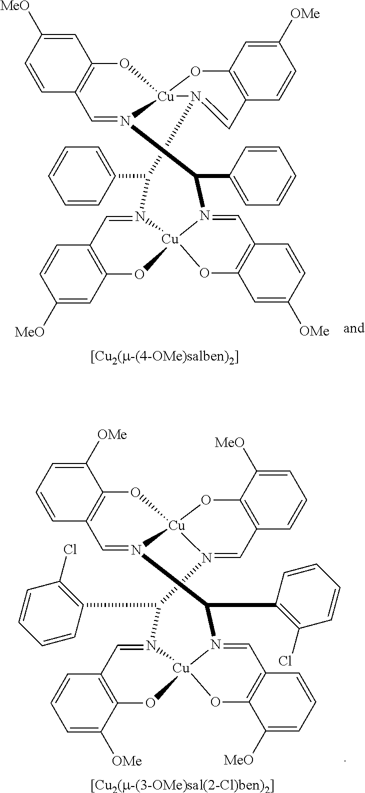

26. The process of claim 22, further comprising contacting the aryl halogenides and N-nucleophile compound with a base, wherein the base is selected from alkali salts of mineral acids, alkali salts of organic acids, and from alkali alcoholates.

27. The process of claim 22, wherein the contacting takes place in the presence of an organic solvent at a reaction temperature between 20 °C and 200 °C.
